(19) United States
(12) Patent Application Publication
(10) Pub. No.: US 2017/0016915 A1
(43) Pub. Date: Jan. 19, 2017

(54) SCREENING TARGETS AND COMPOSITIONS AND METHODS FOR TREATMENT OF CILIOPATHY DISORDERS

(71) Applicant: Duke University, Durham, NC (US)

(72) Inventors: Nicholas Katsanis, Durham, NC (US); Edwin Oh, Durham, NC (US); Yangfan Liu, Durham, NC (US); I-Chun Tsai, Durham, NC (US)

(21) Appl. No.: 15/276,127

(22) Filed: Sep. 26, 2016

Related U.S. Application Data
(63) Continuation of application No. PCT/US15/22327, filed on Mar. 24, 2015.
(60) Provisional application No. 61/969,397, filed on Mar. 24, 2014.

(51) Int. Cl. G01N 33/68; A61K 49/00; C12Q 1/68

(52) U.S. Cl. CPC G01N 33/6893; C12Q 1/6883; A61K 49/0008; G01N 2333/96425; C12Q 2600/136; C12Q 2600/158; G01N 2500/10; G01N 2333/47; C12Q 2600/178

(57) ABSTRACT

The present disclosure provides methods of screening and compositions and methods for treating ciliopathy diseases. Methods are provided for screening for molecules to treat ciliopathy disorders by measuring the activity of ubiquitin-proteasome system (UPS)-mediated protein degradation in the presence and the absence of a candidate molecule, wherein an increase of the activity in the presence of the candidate molecule identifies the molecule as a potential therapeutic. The methods provided also include measuring the activity of a ubiquitin peptidase or a Zic family member 1 (ZIC1) gene product, in the presence and absence of a candidate molecule, wherein a decrease in activity in the presence of the molecule identifies it as a potential therapeutic.

### CROSS REFERENCE TO RELATED APPLICATIONS

This application is a continuation of PCT patent application number PCT/US15/22327 filed Mar. 24, 2015, which claims the benefit of U.S. provisional patent application No. 61/969,397 filed Mar. 24, 2014, the disclosure of which is hereby incorporated by reference in its entirety.

### FEDERAL FUNDING LEGEND

The invention was made with government support under Grant No. R01-HD042601 and Grant No. 1 F32 DK094578-01A1 awarded by the National Institute of Health (NIH). The Government has certain rights in the invention.

### TECHNICAL FIELD

The presently disclosed subject matter relates to screening targets and compositions and methods for treatment of ciliopathy disorders.

### BACKGROUND

The ciliopathies are a group of greater than 100 overlapping clinical disorders caused by defects in the primary cilium and its anchoring structure, the basal body. Since cilia are present on almost all vertebrate cell types, it is not surprising that the cilium modulates tissue and cellular events, including development, homeostasis and even cancer progression. As such, disruption of ciliary function has been shown to cause numerous human genetic disorders, including Polycystic Kidney Disease (PKD), Nephronophthisis (NPH), Bardet-Biedl Syndrome (BBS), Meckel-Gruber Syndrome (MKS), Orofaciodigital Syndrome 1 (OFD1), Joubert Syndrome (JBTS), Jeune Syndrome (JATD), Senior-Loken Syndrome (SLS) and Leber congenital amaurosis (LCA) to name but a few. Consistent with their common organelle defect, these diseases also share phenotypes, including retinal degeneration, polydactyly, cystic kidney disease, situs inversus, mental retardation, cerebellum hypoplasia, hydrometrocolpos, obesity and liver dysfunction.

Even though improved sequencing techniques have facilitated the rate of mutation identification, this group of disorders remains untreatable. So far, no efficient treatment has been designed to ameliorate symptoms or improve disease prognosis. An emerging role of primary cilium is to act as a signaling center facilitating the communication between extra- and intra-cellular signaling effector molecules during development and disease prognosis. To date, the primary cilium has been linked to several signaling pathways that are crucial for proper morphologic development, including Wnt, Shh, and Notch.

In vertebrates, the cilium and the basal body are key components of paracrine signaling transduction. This has, in turn, suggested that phenotypes of ciliopathy patients might be attributed to defective paracrine signaling (1), including polydactyly due to defective sonic hedgehog (Shh) signaling (2) and renal cysts attributed to unbalanced Wnt signaling (3). Some data have raised the possibility that a fraction of these defects, especially Wnt, are driven by nonciliary functions of ciliary and basal body proteins (2, 4); other findings have indicated that the cilium/basal body Wnt roles are likely specific to discrete spatiotemporal contexts (5-9).

Although basal body and ciliary proteins are not signaling molecules per se, these structures are thought to operate as a hub for coordinating networks of signaling cascades. Components of various signaling pathways localize to basal body and cilium (10-13). Moreover, mutations in a single basal body or ciliary gene can lead to defects in more than one signaling pathway (11, 14), while loss-of-function mutations in signaling molecules such as the Shh regulator kinesin family member 7 (KIF7) (15, 16) and the Wnt/planar cell polarity (PCP) effector Fritz (17) cause ciliopathies in some families.

Thus, there remains an unmet need for treatments to ameliorate symptoms or improve disease prognosis for ciliopathic disorders.

### SUMMARY

In one aspect of the presently disclosed subject matter a method is provided for screening for a therapeutic molecule to treat a ciliopathy disorder. The method includes measuring the activity of a ubiquitin-proteasome system (UPS)-mediated protein degradation in the presence and the absence of a candidate molecule, wherein an increase of the UPS-mediated protein degradation activity in the presence of the candidate molecule identifies the candidate molecule as a potential therapeutic molecule to treat a ciliopathy disorder.

In one aspect of the presently disclosed subject matter a method is provided for screening for a therapeutic molecule to treat a ciliopathy disorder. The method includes measuring the activity of a negative regulator of a ubiquitin-proteasome system (UPS) in the presence and the absence of a candidate molecule, wherein a decrease in the activity of the negative regulator of the UPS in the presence of the candidate molecule identifies the candidate molecule as a potential therapeutic molecule to treat a ciliopathy disorder.

In one aspect of the presently disclosed subject matter a method is provided for screening for a therapeutic molecule to treat a ciliopathy disorder. The method includes measuring the activity of a human ubiquitin peptidase (USP35) gene product or an ortholog thereof, in the presence and the absence of a candidate molecule, wherein a decrease of the activity in the presence of the candidate molecule identifies the candidate molecule as a potential therapeutic molecule to treat a ciliopathy disorder.

In one aspect of the presently disclosed subject matter a method is provided for screening for a therapeutic molecule to treat a ciliopathy disorder. The method includes measuring the activity of a Zic family member 1 (ZIC1) gene product or an ortholog thereof, in the presence and the absence of a candidate molecule, wherein a decrease of the activity in the presence of the candidate molecule identifies the candidate molecule as a potential therapeutic molecule to treat a ciliopathy disorder.

In one aspect of the presently disclosed subject matter a small interfering RNA (siRNA) is provided. The siRNA comprises a sense RNA strand and an antisense RNA strand, wherein the sense RNA strand has a sense RNA sequence that is at least 19 nucleotides in length and at least 70% homologous to at least 19 contiguous nucleotides to of USP35 human ubiquitin peptidase cDNA sequence (SEQ ID NO: 1), and wherein the antisense RNA strand has an antisense RNA sequence that is complementary to the sense RNA sequence.

In one aspect of the presently disclosed subject matter a short hairpin RNA (shRNA) is provided. The shRNA comprises a sense RNA sequence, an antisense RNA sequence and a hairpin sequence, wherein the sense RNA sequence is at least 19 nucleotides in length and at least 70% homologous to at least 19 contiguous nucleotides of USP35 human ubiquitin peptidase cDNA sequence (SEQ ID NO: 1), wherein the antisense RNA sequence is at least 19 nucleotides in length and complementary to the sense RNA sequence, and wherein the sense RNA sequence and the antisense RNA sequence are covalently linked by the hairpin sequence.

In one aspect of the presently disclosed subject matter a small interfering RNA (siRNA) is provided. The siRNA comprises a sense RNA strand and an antisense RNA strand, wherein the sense RNA strand has a sense RNA sequence that is from 19 to 29 nucleotides in length and at least 70% homologous to at least 19 contiguous nucleotides of ZIC1 Zic family member 1 cDNA sequence (SEQ ID NO: 2), and wherein the antisense RNA strand has an antisense RNA sequence that is complementary to the sense RNA sequence.

In one aspect of the presently disclosed subject matter a short hairpin RNA (shRNA) is provided. The shRNA comprises a sense RNA sequence, an antisense RNA sequence and a hairpin sequence, wherein the sense RNA sequence is at least 19 nucleotides in length and at least 70% homologous to at least 19 contiguous nucleotides of ZIC1 Zic family member 1 cDNA sequence (SEQ ID NO: 2), wherein the antisense RNA sequence is at least 19 nucleotides in length and complementary to the sense RNA sequence, and wherein the sense RNA sequence and the antisense RNA sequence are covalently linked by the hairpin sequence.

In one aspect of the presently disclosed subject matter a method is provided for treating a ciliopathy disorder, the method includes administering at least one small interfering RNA (siRNA), comprising a sense RNA sequence and an antisense RNA sequence, to an individual having a ciliopathy disorder; and monitoring the level of the ciliopathy disorder, wherein the sense RNA sequence is at least about 70% homologous to at least 19 contiguous nucleotides of a USP35 ubiquitin peptidase cDNA sequence (SEQ ID NO: 1) and wherein the antisense RNA sequence is complementary to the sense RNA sequence.

In one aspect of the presently disclosed subject matter a method is provided for treating a ciliopathy disorder, the method includes administering at least one short hairpin RNA (shRNA), comprising a sense RNA sequence and an antisense RNA sequence covalently linked by a hairpin sequence to an individual having a ciliopathy disorder; and monitoring the level of the ciliopathy disorder, wherein the sense RNA sequence is at least about 70% homologous to at least 19 contiguous nucleotides of a USP35 ubiquitin peptidase cDNA sequence (SEQ ID NO: 1) and wherein the antisense RNA sequence is complementary to the sense RNA sequence.

In one aspect of the presently disclosed subject matter a method is provided for treating a ciliopathy disorder, the method includes administering at least one small interfering RNA (siRNA), comprising a sense RNA sequence and an antisense RNA sequence, to an individual having a ciliopathy disorder; and monitoring the level of the ciliopathy disorder, wherein the sense RNA sequence is at least about 70% homologous to at least 19 contiguous nucleotides of a ZIC1 Zic family member 1 cDNA sequence (SEQ ID NO: 2) and wherein the antisense RNA sequence is complementary to the sense RNA sequence.

In one aspect of the presently disclosed subject matter a method is provided for treating a ciliopathy disorder, the method includes administering at least one small hairpin RNA (shRNA), comprising a sense RNA sequence and an antisense RNA sequence covalently linked by a hairpin sequence, to an individual having a ciliopathy disorder; and monitoring the level of the ciliopathy disorder, wherein the sense RNA sequence is at least about 70% homologous to at least 19 contiguous nucleotides of a ZIC1 Zic family member 1 cDNA sequence (SEQ ID NO: 2) and wherein the antisense RNA sequence is complementary to the sense RNA sequence.

In one aspect of the presently disclosed subject matter a method is provided for screening for a therapeutic molecule to treat a ciliopathy disorder. The method includes determining the ability of a candidate molecule to rescue a defect in a cell-based or an animal-based model, wherein the cell-based or the animal-based model comprises a silenced, reduced, or depleted expression of one or more ciliary genes comprising a BBS4 (Bardet-Biedl syndrome 4) gene, a BBS1 (Bardet-Biedl syndrome 1) gene, or an OFD1 (Oral-facial-digital syndrome 1) gene that results in the defect, and wherein the ability of the candidate molecule to rescue the defect identifies the candidate molecule as a potential therapeutic molecule to treat a ciliopathy disorder.

One aspect of the present disclosure provides a method of treating a ciliopathy disorder in a subject comprising, consisting of, or consisting essentially of administering to the subject a therapeutically effective amount of a proteasome agonist such that the ciliopathy disorder is treated.

### BRIEF DESCRIPTION OF THE DRAWINGS

The foregoing aspects and other features of the invention are explained in the following description, taken in connection with the accompanying drawings.

FIGS. 1A-1C show accumulation of Green Fluorescent Protein (GFR) in Bbs4^{-/-} mice according to one or more embodiments of the present disclosure. (A and B) Immunoblotting with anti-GFP to examine the kidney (P80), liver (P144), several brain components (P80), and retina (P12-P126) (B) of Ub^{G76V}-Gfp Bbs4^{-/-} mice, with Ub^{G76V}-Gfp WT littermates used as controls. Samples in each panel for the brain were run on the same gel but were noncontiguous. (C) Immunohistochemistry of retinal sections of P23 and P126 Ub^{G76V}-Gfp transgenic mice. Progressive retinal degeneration and GFP accumulation in photoreceptors (OS and IS, and ONL) were observed in Bbs4^{-/-} mice, but not in WT littermates. OS was immunolabeled with anti-s-opsin; ONL and INL were labeled with DAPI staining. RPE, retinal pigment epithelium; OS, outer segment of the photoreceptors; IS, inner segment; ONL, outer nuclear layer; OPL, outer plexiform layer; INL, inner nuclear layer; IPL, inner plexiform layer; GCL, ganglion cell layer. White boxes delimit the enlarged images, showing OS and IS. HP, hippocampus; CX, cortex; CB, cerebellum. White boxes delimit the enlarged images, showing OS and IS. Scale bar: 25 μm in images and inserts. Bar graphs showing standard error of the mean are plotted adjacent to each blot. P<0.05; **P<0.01.

FIGS. 2A-2D show accumulation of Shh and Notch signaling mediators upon depletion of ciliopathy proteins according to one or more embodiments of the present disclosure. (A) Accumulation of GLI2F L, GLI3FL, and SUFU as well as reduction of GLI3R in T8-derived Ofd1^{KO} neurons. (B) At E10.5, accumulation of GLI2FL, GLI3FL, and SUFU as well as reduction of GLI3R were detected in protein lysates from Ofd1^{Δ4-5/y} mice, with Ofd1^{+/y} mice used as controls. (C) Suppression of BBS4 increases Flag-tagged NICD levels compared with those in pSuper controls. (D) Suppression of BBS4 led to a 2-fold increase in GFP-tagged JAG1. The samples in each panel in B and C were run on the same gel but were noncontiguous. Bar graphs showing the SEM are plotted adjacent to each blot. *P<0.05; **P<0.01.

FIGS. 3A-3C show disruption of proteasomal degradation caused by loss of ciliopathy proteins according to one or more embodiments of the present disclosure. (A) Treatment of proteasomal agonist sulforaphane (SFN) ameliorated the accumulation of GLI3FL, GLI2FL, and SUFU in T8-derived Ofd1^{KO} neurons. (B) Suppression of BBS4 in HEK-293-FT cells led to a 1.57-fold increase in β-catenin protein levels that could be rescued by SFN. (C) Overexpression of BBS4 reduced NICD levels. MG132 treatment restored Flag-NICD levels. Samples in each panel in C were run on the same gel but were noncontiguous. Bar graphs showing SEM are plotted adjacent to each blot. *P<0.05; **P<0.01; ***P<0.001.

FIGS. 4A-4D show that ciliopathy proteins can interact with proteasomal components and regulate proteasome composition according to one or more embodiments of the present disclosure. (A) Immunoblots show interaction between endogenous OFD1 and Flag-tagged RPT6 and endogenous BBS4 and GFP-tagged RPN10. An endogenous interaction was detected between BBS1 and RPN10 from protein lysate isolated from the testis of C57BL/6 mice. (B) Immunoblots show that suppression of OFD1 in HEK-293 cells reduced RPT2 protein levels in purified 26S proteasome. Densitometry measurements of proteasomal RPT2 protein levels were plotted (n=3), and no significant difference in total RPT2 abundance was detected. Coomassie blue staining was carried out as a loading control and to measure the efficiency of the 26S proteasomal purification process. (C) Suppression of OFD1 in HEK-293 cells resulted in the reduction of pericentriolar RPN10 levels (normalized to cytoplasmic RPN10 levels) in OFD1-depleted cells. Scale bar: 10 μm. (D) HEK-293-FT cells transfected with pSuper control and pSuperOFD1 were subjected to sucrose gradient centrifugation. Fractions (fractions 6-12 of 13 fractions) were then analyzed by immunoblotting with antibodies against γ-tubulin and proteasomal subunits. In control cells, the fractions enriched with all proteasome subunits partially overlapped with fractions enriched with γ-tubulin (fraction 8), and when OFD1 was depleted, peak levels of RPN10, RPN13, RPT2, and RPT6 shifted significantly and resulted in a decrease in the overlap between γ-tubulin-enriched fractions and 19S subunit-enriched fractions. **P<0.01; ***P<0.001.

FIGS. 5A-5D show that activation of the proteasome can ameliorate signaling defects in bbs and ofd1 morphant zebrafish embryos according to one or more embodiments of the present disclosure. (A) Coinjection of human RPN10, RPN13, and RPT6 mRNA into bbs4 and ofd1 morphant zebrafish embryos rescued somitic and CE (convergent extension) defects at the 9±1 ss and ectopic expression of her4 in the eye (arrowheads in lower row) at 4.5 dpf. CE defects were scored based on the body gap angle (arrowheads in upper row). Expression of her4 was detected by whole-mount RNA in situ hybridization. (B, C, and D) Coinjection of SFN rescued somitic and CE defects as well as ectopic her4 expression in bbs4 (B), bbs1 (C), and ofd1 (D) morphant zebrafish embryos, while injection of SFN alone did not give rise to any obvious phenotype (B). As shown in the top row (dorsal view), the somites (bars) were longer in bbs4 (B), bbs1 (C), and ofd1 (D) morphants and were shortened in morphant zebrafish embryos coinjected with SFN. In the second row (lateral view), the body gap angle (arrowheads) was greater in morphants and reduced in the presence of SFN. Dashed boxes delimit the enlarged images in the third row, showing the effects of SFN treatment on somite boundary definition defects. Percentage of embryos with somite boundary definition and CE defects and sample size (n) are noted below the images of each condition. Scale bars: 100 μm.

FIGS. 6A-6G show NF-κB signaling defects in BBS4-, BBS1-, and OFD1-depleted cells, and Ofd1 conditional knockout mice according to one or more embodiments of the present disclosure. (A-C) Suppression of BBS4 (A), BBS1 (B), or OFD1 (C) in HEK-293-FT cells led to decreased 3X-κB-L luciferase reporter responsiveness to a 12-hour TNF-α treatment. Incubation with SFN for 6 hours partially restored sensitivity to TNF-α stimulation (n=3). (D and E) GFP-tagged IκBβ protein levels were higher in HEK-293-FT cells depleted of BBS4, BBS1 (D), or OFD1 (E) compared with those in control. SFN incubation rescued IκBα-GFP accumulation. (F) Protein levels of IκBβ in kidney tissues isolated from Ofd1^{fl/y} CAG-Cre-ER™ (Ofd1^{fl/y} CKO) mice at P8 (precystic stage) and P20 (cystic stage) were higher than the levels in kidney tissues from WT littermates. (G) mRNA levels of kBP (Nfkbib) in kidney tissues were lower in Ofd1^{fl/y} CKO compared with mRNA levels in WT animals at P8 and were comparable between Ofd1^{fl/y} CAG-Cre-ER™ and controls at P20 (n=3). Bar graphs showing SEM are plotted adjacent to each blot. *P<0.05; **P<0.01; ***P<0.001.

FIG. 7 shows a network of co-expressed ciliary genes according to one or more embodiments of the present disclosure. In silico analysis of co-expressed ciliary genes reveals a subset of transcripts, including BBS1, BBS4 and OFD1.

FIG. 8 shows a schematic of a timeline of in vitro induced differentiation of neurons from embryonic stem cells according to one or more embodiments of the present disclosure. Mouse embryonic stem cells (ESs) were cultured to generate neural subtypes. Four to six days after induction, an enriched population of neuroepithelial precursors is formed. During day 7-8, neuroepithelial precursors are organized into characteristic rosette-like structures, in a process that resembles neural tube formation in the embryo. At day 8-10, differentiating neurons lose contact with the center of rosettes and migrate to their periphery.

FIGS. 9A-9F show how a loss of ciliopathy proteins disrupts proteasomal degradation of Shh and Notch signaling mediators according to one or more embodiments of the present disclosure. (A) Verification of OFD1 protein levels in Ofd1^{Δ4-5/y} knockout mice. (B-D) GLI3R levels are reduced in both T8 derived neurons and in tissue from Ofd1^{Δ4-5/y} knockout mice. (E) Overexpression of BBS4 reduces NICD (both endogenous NICD and exogenous Flag-NICD), and lactacystin treatment increases NICD levels. (F) Overexpression of BBS4 reduces GFP-JAG1 levels to 0.25 fold of cells expressing endogenous BBS4. MG132 treatment ameliorates the BBS4 overexpression phenotype. Bar graphs showing the SEM are plotted adjacent to each blot. *P<0.05; **P<0.01; ***P<0.001.

FIGS. 10A-10C show that BBS proteins interact with proteasomal components and regulate proteasome composition according to one or more embodiments of the present disclosure. (A) Immunoprecipitation shows interaction between several Myc- or HA-tagged BBS proteins (BBS1, 2, 4, 6, 7, and 8) and multiple GFP-tagged proteasomal subunits (PSMB1, RPN10, RPN13, RPT6, and PA28γ. The asterisks indicate non-specific detection of heavy chain and light chain. (B) Suppression of BBS4 in HEK-293-FT cells reduces proteasomal RPN10 protein levels, but not the protein levels of total RPN10 in the cells. Coomassie blue staining show even loading equal efficiency of the 26S proteasome purification in different samples. (C) Distribution of proteasome subunits relative to α-tubulin in sucrose gradient centrifuge fractions. When BBS4 were depleted in HEK-293-FT cells, peak levels of RPN10, RPN13, RPT2, RPT6 shifted and the overlap with α-tubulin-enriched fractions decreased. Bar graphs showing the SEM are plotted adjacent to each blot. **P<0.01.

FIGS. 11A-11E show somite boundary definition defects and persistent her4 expression in bbs morphant embryos according to one or more embodiments of the present disclosure. (A) Representative examples of control zebrafish embryos displaying distinct, straight somite boundaries, compared to bbs1 and bbs4 morphant zebrafish embryos. Dashed boxes in top panel denote the enlarged images in bottom panel. The percentage of embryos with somite boundary definition defects and the sample size (n) are noted below the image of each condition. (B) Representative examples of the expression domain of Notch target gene her4, assessed by whole-mount RNA in situ hybridization in control embryos and bbs4 morphants. Expansion of the her4 expression domain can be observed along the antero-posterior midline in bbs4 morphant zebrafish embryos. Lateral views, anterior at the top. (C) The proportion of embryos with and without expansion of expression domain are noted along the y-axis. Sample size (n) is noted for each group. (D,E) Whole-mount RNA in situ hybridization of her4 at a series of development stages from 1.5 dpf to 5 dpf. Expression of her4 in the developing neural structures of the head, especially in the eye (arrowheads), begins to wane in control embryos after 3 days post fertilization (dpf), but persists in bbs4 morphants through 5 dpf. Scale bar: 100 μm.

FIG. 12 shows MVA treatment of bbs4 morpant zebrafish rescues Wnt and Notch signaling defects Co-injection of MVA rescues somitic and CE defects, as well as ectopic her4 expression in bbs4 morphant zebrafish embryos according to one or more embodiments of the present disclosure. Scale bar: 100 μm.

FIGS. 13A-13C show a genome-wide siRNA screening to identify functional suppressors of BBS4 according to one or more embodiments of the present disclosure. (A) Experimental design of genome-wide siRNA screening. (B) Results of primary screening and secondary validation. (C) Timeline of in vivo assays. Morpholinos targeting to bbs4 or suppressor genes were injected in 1-2 cells per stage. The CE, cerebellum and renal developments were assessed at the time points as indicated in the FIG.

FIGS. 14A-14C show use of a zebrafish model demonstrating the rescue efficacy of usp38 suppression. (A) Depletion of bbs4 results in CE defects, including wider anterior-posterior body gap, somite (Class I) and loss of eyes (Class II). Co-injection of usp38-MO reduces both Class I and Class II embryos. (***: p<0.001). (B) (C) Knock-down of usp38 abundance ameliorates cerebellum (B) and renal (C) defects seen in bbs4 morphant.

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications to of the disclosure as illustrated herein, being contemplated as would normally occur to one skilled in the art to which the disclosure relates.

The presently disclosed subject matter provides a common mechanism for the multiple divergent pathways for ciliary and basal body protein involvement that have been shown to be associated with signaling defects. In one aspect of the present disclosure, the ubiquitin-proteasome system (UPS) is provided as a common mechanism and a target for treatment of ciliopathy disorders. First, it was noted that the basal body is known to be a proteolytic center (18-21). Second, it was noted that previous reports indicate that disruption of some basal body proteins result in loss of proteasome-dependent degradation of β-catenin (5), a phenotype reproduced subsequently (22). Finally, it was noted that, in addition to Wnt, proteasomal degradation is implicated in most paracrine signaling cascades known to be defective in basal body mutants.

For example, in Notch-mediated lateral inhibition, a transmembrane ligand of the Delta-Serrate-LAG2 (DSL) family binds a transmembrane Notch receptor in the adjacent target cells (23). To reduce Notch signaling, DSL ligands are ubiquitinated, internalized, and degraded by the proteasome (24); similarly, the intracellular domain of the Notch receptor is degraded by the proteasome to reduce Notch signaling (25). Likewise, when Shh ligand is present, glioma-associated oncogenes 2 and 3 (GLI2/3) exist in their full-length activator forms, whereas they are truncated by proteasome-mediated proteolysis to their repressor forms when Shh is removed (26); both the activator and repressor forms of GLI2/3 are also degraded by the proteasome (27). Besides GLI2/3, suppressor of fused homolog (SUFU), a negative regulator of Shh signaling that physically localizes at the cilium, is also degraded in a proteasome-dependent manner (28). Aspects of the present disclosure provide that basal body and ciliary proteins can regulate multiple signaling pathways by controlling proteasome-mediated degradation of signaling mediators.

Aspects of the presently disclosed subject matter demonstrate that suppression of basal body-localized ciliopathy proteins can lead to defective proteasomal degradation of such mediators, which in turn can cause dysfunction in three major cilia-associated signaling pathways (Shh, Wnt, and Notch) in vitro and in vivo. These results are unlikely to reflect nonspecific cellular malaise; as not only could the ciliopathy proteins tested herein interact with specific regulatory subunits of the proteasome holoenzyme, but also depletion of each of the ciliopathy proteins Bardet-Biedl syndrome 4 (BBS4) and oral-facial-digital syndrome 1 (OFD1) selectively perturbed the subunit composition of the centrosomal proteasome.

More specifically, the presently disclosed subject matter demonstrates that loss of cilopathy-associated proteins Bardet-Biedl syndrome 4 (BBS4) or oral-facial-digital syndrome 1 (OFD1) results in the accumulation of signaling mediators normally targeted for proteasomal degradation. In wild-type (WT) cells, several BBS proteins and OFD1 interacted with proteasomal subunits, and loss of either BBS4 or OFD1 led to depletion of multiple subunits from the centrosomal proteasome. Furthermore, overexpression of proteasomal regulatory components or treatment with proteasomal activators sulforaphane (SFN) and mevalonolactone (MVA) ameliorated signaling defects in cells lacking BBS1, BBS4, and OFD1, in morphant zebrafish embryos, and in induced neurons from Ofd1-deficient mice.

In addition, the hypothesis that other proteasome-dependent pathways not known to be associated with ciliopathies are defective in the absence of ciliopathy proteins was tested in the presently disclosed subject matter. It was discovered that loss of BBS1, BBS4, or OFD1 led to decreased NF-κB activity and concomitant IκBβ accumulation and that these defects were ameliorated with SFN treatment. Taken together, these data indicate that basal body proteasomal regulation can govern paracrine signaling pathways and indicate that augmenting proteasomal function can benefit ciliopathy patients.

Furthermore, in a separate approach from the experiments described above, a genome-wide siRNA screening assay was performed in a human cell line model of loss of ciliary function to identify genes whose suppression can rescue the aberrant signaling transduction caused by loss of ciliary function. The siRNA screening assay and the resulting data are described in the present disclosure. In this siRNA assay, USP35, a deubiquitinase (ubiquitin peptidase) that acts as a negative regulator of the ubiquitin-proteasome-system (UPS), was identified, as well as 12 other gene targets for ciliopathy disorders. The veracity of the observed rescue effect seen in the cell model was then further tested in an animal model of ciliopathy disorders for 6 of the 13 identified targets. Specifically, a zebrafish model was used to recapitulate pathogenic features in patients. Depletion of the gene product for 5 of the 6 identified targets in the zebrafish model, including USP35, was demonstrated to ameliorate the cerebellar and renal defects in the ciliary deficient animal model.

Thus, one aspect of the present disclosure provides methods for benefiting ciliopathy patients by increasing UPS-mediated protein degradation activity.

Articles “a” and “an” are used herein to refer to one or to more than one (i.e. at least one) of the grammatical object of the article. By way of example, “an element” means at least one element and can include more than one element.

Unless otherwise defined, all technical terms used herein have the same meaning as commonly understood by one of ordinary skill in the art to which this disclosure belongs.

As used herein, the term “ciliopathy disorder”, “ciliopathy disease”, “ciliopathic disease”, “ciliopathies” and ciliopathic disorders” are used interchangeably and refer to those genetic disorders of the cellular cilia, the cilia anchoring structures, the basal bodies, and/or ciliary function. Examples of such disorder include, but are not limited to, Alstrom Syndrome, Bardet-Biedl Syndrome (BBS) (e.g., BBS1, BBS2, BBS4, BBS5, BBS7, BBS9, BBS10, BBS12, ARL6, MKKS, TTC8, TRIM32), Joubert Syndrome, Meckel-Gruber syndrome, Nephronophthisis, Oral-facial-digital syndrome 1 (OFD1), Senior-Loken Syndrome, Polycystic kidney disease, primary ciliary dyskinsesia, asphyxiating thoracic dysplasia, Marden-Walker syndrome, situs inversus/Isomerism, and the like.

As used herein the term “ubiquitin-proteasome system (UPS)” is used interchangeably with the term “ubiquitin-proteasome pathway” or the term “proteasome pathway”. As used herein the term “proteasome agonist” refers to any compound or molecule that is capable of activating the proteasome pathway, either by activating the proteasome itself, or a protein within or associated with the proteasome pathway that results in the activation of the proteasome. In one example a protein that is within or associated with the proteasome pathway is a negative regulator of the proteosome pathway. Examples of proteosome agonists include, but are not limited to, sulforaphane (SFN; 1-isothiocyanato-4(R)-methylsulfinylbutane) and mevalonolactone (MVA).

As used herein, the term “subject” and “patient” and “individual” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, mice, chickens, amphibians, reptiles, and the like. Preferably, the subject is a human patient. More preferably, the subject is a human patient suffering from a ciliopathy disorder.

As used herein, “treatment” is a clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition. “Treatments” refer to one or both of therapeutic treatment and prophylactic or preventative measures. Those in need of treatment include those already affected by a disease or disorder or undesired physiological condition as well as those in which the disease or disorder or undesired to physiological condition is to be prevented.

The term “effective amount” or “therapeutically effective amount” refers to an amount sufficient to effect beneficial or desirable biological and/or clinical results.

In one aspect of the presently disclosed subject matter a method is provided for screening for a therapeutic molecule to treat a ciliopathy disorder. The method includes measuring the activity of a ubiquitin-proteasome system (UPS)-mediated protein degradation in the presence and the absence of a candidate molecule, wherein an increase of the UPS-mediated protein degradation activity in the presence of the candidate molecule identifies the candidate molecule as a potential therapeutic molecule to treat a ciliopathy disorder. The UPS-mediated protein degradation activity can be measured directly at the ubiquitin-proteasome holoenzyme. The candidate molecule can include a small molecule or an antibody.

The UPS-mediated protein degradation activity can be measured indirectly via a cell-based or an animal-based model, wherein the cell-based or the animal-based model has a silenced, reduced, or depleted expression of one or more ciliary genes having a BBS4 (Bardet-Biedl syndrome 4) gene, a BBS1 (Bardet-Biedl syndrome 1) gene, or an OFD1 (Oral-facial-digital syndrome 1) gene that results in reduced ubiquitin-proteasome system (UPS)-mediated protein degradation. The candidate molecule can include a small molecule, an antibody, a RNA interference molecule (RNAi), a short hairpin RNA (shRNA), or a small interfering RNA (siRNA).

The cell-based model can include a human retinal pigmentosa epicedium cell line (RPE). The ciliary gene can be the BBS4 gene. The RPE cell line can stably expresses a short hairpin RNA (shRNA) against BBS4 expression.

The animal-based model can include a morphant zebrafish embryo model having a bbs4 depletion-induced convergent extension (CE) defect, a cerebellum organizational abnormality, and a renal development abnormality. The animal-based model can include an Ofd1 knockout mouse model.

In one aspect of the presently disclosed subject matter a method is provided for screening for a therapeutic molecule to treat a ciliopathy disorder. The method includes measuring the activity of a negative regulator of a ubiquitin-proteasome system (UPS) in the presence and the absence of a candidate molecule, wherein a decrease in the activity of the negative regulator of the UPS in the presence of the candidate molecule identifies the candidate molecule as a potential therapeutic molecule to treat a ciliopathy disorder.

The negative regulator of the ubiquitin-proteasome system (UPS) can include: a ubiquitin peptidase (USP35) gene product or an ortholog thereof, a Zic family member 1 (ZIC1) gene product or an ortholog thereof, a dopamine receptor D5 (DRD5) gene product or an ortholog thereof, a prothymosin alpha gene sequence 28 (PTMA) or an ortholog thereof, an endo-beta-N-acetylglucosaminidase (ENGASE) gene product or an ortholog thereof, a phosphatidylinositol transfer protein (PITPNM2) gene product or an ortholog thereof, a Rhox homeobox family member 1 (RHOXF1) gene product or an ortholog thereof, an ectonucleoside triphosphate diphosphohydrolase 6 (ENTPD6) gene product or an ortholog thereof, a chromosome 14 open reading frame 166 (C14orf166) gene product or an ortholog thereof, a cleavage and polyadenylation factor subunit homolog (PCF11) gene product or an ortholog thereof, a testis expressed 36 (TEX36) gene product or an ortholog thereof, or a tudor domain containing 12 (TDRD12) gene product or an ortholog thereof.

In the method for screening for a therapeutic molecule to treat a ciliopathy disorder, the candidate molecule can include a small molecule, an antibody, a RNA interference molecule (RNAi), a short hairpin RNA (shRNA), or a small interfering RNA (siRNA).

In one aspect of the presently disclosed subject matter a method is provided for screening for a therapeutic molecule to treat a ciliopathy disorder. The method includes measuring the activity of a ubiquitin peptidase USP35 gene product, or an ortholog to thereof, in the presence and the absence of a candidate molecule, wherein a decrease of the activity in the presence of the candidate molecule identifies the candidate molecule as a potential therapeutic molecule to treat a ciliopathy disorder.

In one aspect of the presently disclosed subject matter a method is provided for screening for a therapeutic molecule to treat a ciliopathy disorder. The method includes measuring the activity of a Zic family member 1 (ZIC1) gene product or an ortholog thereof, in the presence and the absence of a candidate molecule, wherein a decrease of the activity in the presence of the candidate molecule identifies the candidate molecule as a potential therapeutic molecule to treat a ciliopathy disorder.

In one aspect of the presently disclosed subject matter a method is provided for screening for a therapeutic molecule to treat a ciliopathy disorder. The method includes measuring the activity of an endo-beta-N-acetylglucosaminidase (ENGASE) gene product, or an ortholog thereof, in the presence and the absence of a candidate molecule, wherein a decrease of the activity in the presence of the candidate molecule identifies the candidate molecule as a potential therapeutic molecule to treat a ciliopathy disorder.

In one aspect of the presently disclosed subject matter a method is provided for screening for a therapeutic molecule to treat a ciliopathy disorder. The method includes measuring the activity of a membrane-associated 2 phosphatidylinositol transfer protein (PITPNM2) gene product, or an ortholog thereof, in the presence and the absence of a candidate molecule, wherein a decrease of the activity in the presence of the candidate molecule identifies the candidate molecule as a potential therapeutic molecule to treat a ciliopathy disorder.

In one aspect of the presently disclosed subject matter a method is provided for screening for a therapeutic molecule to treat a ciliopathy disorder. The method includes measuring the activity of a dopamine receptor D5 (DRD5) gene product or an ortholog thereof, in the presence and the absence of a candidate molecule, wherein a decrease of the activity in the presence of the candidate molecule identifies the candidate molecule as a potential therapeutic molecule to treat a ciliopathy disorder.

In one aspect of the presently disclosed subject matter a method is provided for screening for a therapeutic molecule to treat a ciliopathy disorder. The method includes measuring the activity of a Rhox homeobox family member 1 (RHOXF1) gene product or an ortholog thereof, in the presence and the absence of a candidate molecule, wherein a decrease of the activity in the presence of the candidate molecule identifies the candidate molecule as a potential therapeutic molecule to treat a ciliopathy disorder.

In one aspect of the presently disclosed subject matter a method is provided for screening for a therapeutic molecule to treat a ciliopathy disorder. The method includes measuring the activity of an ectonucleoside triphosphate diphosphohydrolase 6 (ENTPD6) gene product or an ortholog thereof, in the presence and the absence of a candidate molecule, wherein a decrease of the activity in the presence of the candidate molecule identifies the candidate molecule as a potential therapeutic molecule to treat a ciliopathy disorder.

In one aspect of the presently disclosed subject matter a method is provided for screening for a therapeutic molecule to treat a ciliopathy disorder. The method includes measuring the activity of a chromosome 14 open reading frame 166 (C14orf166) gene product or an ortholog thereof, in the presence and the absence of a candidate molecule, wherein a decrease of the activity in the presence of the candidate molecule identifies the candidate molecule as a potential therapeutic molecule to treat a ciliopathy disorder.

In one aspect of the presently disclosed subject matter a method is provided for screening for a therapeutic molecule to treat a ciliopathy disorder. The method includes measuring the activity of a cleavage and polyadenylation factor subunit homolog (PCF11) gene product or an ortholog thereof, in the presence and the absence of a candidate molecule, wherein a decrease of the activity in the presence of the candidate molecule identifies the candidate molecule as a potential therapeutic molecule to treat a ciliopathy disorder.

In one aspect of the presently disclosed subject matter a method is provided for screening for a therapeutic molecule to treat a ciliopathy disorder. The method includes measuring the activity of a testis expressed 36 (TEX36) gene product or an ortholog thereof, in the presence and the absence of a candidate molecule, wherein a decrease of the activity in the presence of the candidate molecule identifies the candidate molecule as a potential therapeutic molecule to treat a ciliopathy disorder.

In one aspect of the presently disclosed subject matter a method is provided for screening for a therapeutic molecule to treat a ciliopathy disorder. The method includes measuring the activity of a tudor domain containing 12 (TDRD12) gene product or an ortholog thereof, in the presence and the absence of a candidate molecule, wherein a decrease of the activity in the presence of the candidate molecule identifies the candidate molecule as a potential therapeutic molecule to treat a ciliopathy disorder.

In one aspect of the presently disclosed subject matter a small interfering RNA (siRNA) is provided. The siRNA comprises a sense RNA strand and an antisense RNA strand, wherein the sense RNA strand has a sense RNA sequence that is at least 19 nucleotides in length and at least 70% homologous to at least 19 contiguous nucleotides of USP35 human ubiquitin peptidase cDNA sequence (SEQ ID NO: 1), and wherein the antisense RNA strand has an antisense RNA sequence that is complementary to the sense RNA sequence.

In one aspect of the presently disclosed subject matter a short hairpin RNA (shRNA) is provided. The shRNA comprises a sense RNA sequence, an antisense RNA sequence and a hairpin sequence, wherein the sense RNA sequence is at least 19 nucleotides in length and at least 70% homologous to at least 19 contiguous nucleotides of USP35 human ubiquitin peptidase cDNA sequence (SEQ ID NO: 1), wherein the antisense RNA sequence is at least 19 nucleotides in length and complementary to the sense RNA sequence, and wherein the sense RNA sequence and the antisense RNA sequence are covalently linked by the hairpin sequence.

In one aspect of the presently disclosed subject matter a small interfering RNA (siRNA) is provided. The siRNA comprises a sense RNA strand and an antisense RNA strand, wherein the sense RNA strand has a sense RNA sequence that is from 19 to 29 nucleotides in length and at least 70% homologous to at least 19 contiguous nucleotides of ZIC1 Zic family member 1 cDNA sequence (SEQ ID NO: 2), and wherein the antisense RNA strand has an antisense RNA sequence that is complementary to the sense RNA sequence.

In one aspect of the presently disclosed subject matter a short hairpin RNA (shRNA) is provided. The shRNA comprises a sense RNA sequence, an antisense RNA sequence and a hairpin sequence, wherein the sense RNA sequence is at least 19 nucleotides in length and at least 70% homologous to at least 19 contiguous nucleotides of ZIC1 Zic family member 1 cDNA sequence (SEQ ID NO: 2), wherein the antisense RNA sequence is at least 19 nucleotides in length and complementary to the sense RNA sequence, and wherein the sense RNA sequence and the antisense RNA sequence are covalently linked by the hairpin sequence.

In one aspect of the presently disclosed subject matter a small interfering RNA (siRNA) is provided. The siRNA comprises a sense RNA strand and an antisense RNA strand, wherein the sense RNA strand has a sense RNA sequence that is at least 19 nucleotides in length, wherein the antisense RNA strand has an antisense RNA sequence that is complementary to the sense RNA sequence, and wherein the sense RNA sequence comprises: at least 70% homology to at least 19 contiguous nucleotides of a DRD5 dopamine receptor D5 cDNA sequence (SEQ ID NO: 3); at least 70% homology to at least 19 contiguous nucleotides of a PTMA prothymosin alpha gene sequence 28 cDNA sequence (SEQ ID NO: 4); at least 70% homology to at least 19 contiguous nucleotides of a ENGASE endo-beta-N-acetylglucosaminidase cDNA sequence (SEQ ID NO: 5); at least 70% homology to at least 19 contiguous nucleotides of a PITPNM2 phosphatidylinositol transfer protein cDNA sequence (SEQ ID NO: 6); at least 70% homology to at least 19 contiguous nucleotides of a RHOXF1 Rhox homeobox family member 1 cDNA sequence (SEQ ID NO: 7); at least 70% homology to at least 19 contiguous nucleotides of a ENTPD6 ectonucleoside triphosphate diphosphohydrolase 6 cDNA sequence (SEQ ID NO: 8); at least 70% homology to at least 19 contiguous nucleotides of a C14orf166 chromosome 14 open reading frame 166 cDNA sequence (SEQ ID NO: 9); at least 70% homology to at least 19 contiguous nucleotides of a PCF11 cleavage and polyadenylation factor subunit homolog cDNA sequence (SEQ ID NO: 10); at least 70% homology to at least 19 contiguous nucleotides of a TEX36 testis expressed 36 cDNA sequence (SEQ ID NO: 11); or at least 70% homology to at least 19 contiguous nucleotides of a TDRD12 tudor domain containing 12 cDNA sequence (SEQ ID NO: 12).

In one aspect of the presently disclosed subject matter a short hairpin RNA (shRNA) is provided. The shRNA comprises a sense RNA sequence, an antisense RNA sequence and a hairpin sequence, wherein the sense RNA sequence is at least 19 nucleotides in length, wherein the antisense RNA sequence is at least 19 nucleotides in length and complementary to the sense RNA sequence, wherein the sense RNA sequence and the antisense RNA sequence are covalently linked by the hairpin sequence, and wherein the sense RNA sequence comprises: at least 70% homology to at least 19 contiguous nucleotides of a DRD5 dopamine receptor D5 cDNA sequence (SEQ ID NO: 3); at least 70% homology to at least 19 contiguous nucleotides of a PTMA prothymosin alpha gene sequence 28 cDNA sequence (SEQ ID NO: 4); at least 70% homology to at least 19 contiguous nucleotides of a ENGASE endo-beta-N-acetylglucosaminidase cDNA sequence (SEQ ID NO: 5); at least 70% homology to at least 19 contiguous nucleotides of a PITPNM2 phosphatidylinositol transfer protein cDNA sequence (SEQ ID NO: 6); at least 70% homology to at least 19 contiguous nucleotides of a RHOXF1 Rhox homeobox family member 1 cDNA sequence (SEQ ID NO: 7); at least 70% homology to at least 19 contiguous nucleotides of a ENTPD6 ectonucleoside triphosphate diphosphohydrolase 6 cDNA sequence (SEQ ID NO: 8 at least 70% homology to at least 19 contiguous nucleotides of a C14orf166 chromosome 14 open reading frame 166 cDNA sequence (SEQ ID NO: 9); at least 70% homology to at least 19 contiguous nucleotides of a PCF11 cleavage and polyadenylation factor subunit homolog cDNA sequence (SEQ ID NO: 10); at least 70% homology to at least 19 contiguous nucleotides of a TEX36 testis expressed 36 cDNA sequence (SEQ ID NO: 11); or at least 70% homology to at least 19 contiguous nucleotides of a TDRD12 tudor domain containing 12 cDNA sequence (SEQ ID NO: 12).

In one aspect of the presently disclosed subject matter methods are provided for treating a ciliopathy disorder by administering a small interfering RNA (siRNA) or a short hairpin RNA (shRNA) to an individual having a ciliopathy disorder. The siRNA's and shRNA's can be administered as a pharmaceutical composition in combination with a pharmaceutically acceptable carrier. The siRNA's and shRNA's can be administered in combination with a delivery reagent.

In one aspect of the presently disclosed subject matter a method is provided for treating a ciliopathy disorder, the method includes administering at least one small interfering RNA (siRNA), comprising a sense RNA sequence and an antisense RNA sequence, to an individual having a ciliopathy disorder; and monitoring the level of the ciliopathy disorder, wherein the sense RNA sequence is at least about 70% homologous to at least 19 contiguous nucleotides of a USP35 ubiquitin peptidase cDNA sequence (SEQ ID NO: 1) and wherein the antisense RNA sequence is complementary to the sense RNA sequence.

In one aspect of the presently disclosed subject matter a method is provided for treating a ciliopathy disorder, the method includes administering at least one short hairpin RNA (shRNA), comprising a sense RNA sequence and an antisense RNA sequence covalently linked by a hairpin sequence to an individual having a ciliopathy disorder; and monitoring the level of the ciliopathy disorder, wherein the sense RNA sequence is at least about 70% homologous to at least 19 contiguous nucleotides of a USP35 ubiquitin peptidase cDNA sequence (SEQ ID NO: 1) and wherein the antisense RNA sequence is complementary to the sense RNA sequence.

In one aspect of the presently disclosed subject matter a method is provided for treating a ciliopathy disorder, the method includes administering at least one small interfering RNA (siRNA), comprising a sense RNA sequence and an antisense RNA sequence, to an individual having a ciliopathy disorder; and monitoring the level of the ciliopathy disorder, wherein the sense RNA sequence is at least about 70% homologous to at least 19 contiguous nucleotides of a ZIC1 Zic family member 1 cDNA sequence (SEQ ID NO: 2) and wherein the antisense RNA sequence is complementary to the sense RNA sequence.

In one aspect of the presently disclosed subject matter a method is provided for treating a ciliopathy disorder, the method includes administering at least one small hairpin RNA (shRNA), comprising a sense RNA sequence and an antisense RNA sequence covalently linked by a hairpin sequence, to an individual having a ciliopathy disorder; and monitoring the level of the ciliopathy disorder, wherein the sense RNA sequence is at least about 70% homologous to at least 19 contiguous nucleotides of a ZIC1 Zic family member 1 cDNA sequence (SEQ ID NO: 2) and wherein the antisense RNA sequence is complementary to the sense RNA sequence.

In one aspect of the presently disclosed subject matter a method is provided for treating a ciliopathy disorder, the method includes administering at least one small interfering RNA (siRNA), comprising a sense RNA sequence and an antisense RNA sequence, and wherein the antisense RNA sequence is complementary to the sense RNA sequence, to an individual having a ciliopathy disorder; and monitoring the level of the ciliopathy disorder, wherein the sense RNA sequence is at least about 70% homologous to at least 19 contiguous nucleotides of a cDNA sequence comprising: a cDNA sequence comprising a DRD5 dopamine receptor D5 cDNA sequence (SEQ ID NO: 3); a cDNA sequence comprising a PTMA prothymosin alpha gene sequence 28 cDNA sequence (SEQ ID NO: 4); a cDNA sequence comprising an ENGASE endo-beta-N-acetylglucosaminidase cDNA sequence (SEQ ID NO: 5); a cDNA sequence comprising a PITPNM2 phosphatidylinositol transfer protein cDNA sequence (SEQ ID NO: 6); a cDNA sequence comprising a RHOXF1 Rhox homeobox family member 1 cDNA sequence (SEQ ID NO: 7); a cDNA sequence comprising an ENTPD6 ectonucleoside triphosphate diphosphohydrolase 6 cDNA sequence (SEQ ID NO: 8); a cDNA sequence comprising a C14orf166 chromosome 14 open reading frame 166 cDNA sequence (SEQ ID NO: 9); a cDNA sequence comprising a PCF11 cleavage and polyadenylation factor subunit homolog cDNA sequence (SEQ ID NO: 10); a cDNA sequence comprising a TEX36 testis expressed 36 cDNA sequence (SEQ ID NO: 11); or a cDNA sequence comprising a TDRD12 tudor domain containing 12 cDNA sequence (SEQ ID NO: 12).

In one aspect of the presently disclosed subject matter a method is provided for treating a ciliopathy disorder, the method includes administering at least one short hairpin RNA (shRNA), comprising a sense RNA sequence and an antisense RNA sequence covalently linked by a hairpin sequence, and wherein the antisense RNA sequence is complementary to the sense RNA sequence, to an individual having a ciliopathy disorder; and monitoring the level of the ciliopathy disorder, wherein the sense RNA sequence is at least about 70% homologous to at least 19 contiguous nucleotides of a cDNA sequence comprising: a cDNA sequence comprising a DRD5 dopamine receptor D5 cDNA sequence (SEQ ID NO: 3); a cDNA sequence comprising a PTMA prothymosin alpha gene sequence 28 cDNA sequence (SEQ ID NO:4); a cDNA sequence comprising an ENGASE endo-beta-N-acetylglucosaminidase cDNA sequence (SEQ ID NO: 5); a cDNA sequence comprising a PITPNM2 phosphatidylinositol transfer protein cDNA sequence (SEQ ID NO: 6); a cDNA sequence comprising a RHOXF1 Rhox homeobox family member 1 cDNA sequence (SEQ ID NO: 7); a cDNA sequence comprising an ENTPD6 ectonucleoside triphosphate diphosphohydrolase 6 cDNA sequence (SEQ ID NO: 8); a cDNA sequence comprising a C14orf166 chromosome 14 open reading frame 166 cDNA sequence (SEQ ID NO: 9); a cDNA sequence comprising a PCF11 cleavage and polyadenylation factor subunit homolog cDNA sequence (SEQ ID NO: 10); a cDNA sequence comprising a TEX36 testis expressed 36 cDNA sequence (SEQ ID NO: 11); or a cDNA sequence comprising a TDRD12 tudor domain containing 12 cDNA sequence (SEQ ID NO: 12).

In one aspect of the presently disclosed subject matter a method is provided for screening for a therapeutic molecule to treat a ciliopathy disorder. The method includes determining the ability of a candidate molecule to rescue a defect in a cell-based or an animal-based model, wherein the cell-based or the animal-based model comprises a silenced, reduced, or depleted expression of one or more ciliary genes comprising a BBS4 (Bardet-Biedl syndrome 4) gene, a BBS1 (Bardet-Biedl syndrome 1) gene, or an OFD1 (Oral-facial-digital syndrome 1) gene that results in the defect, and wherein the ability of the candidate molecule to rescue the defect identifies the candidate molecule as a potential therapeutic molecule to treat a ciliopathy disorder.

In the method for screening for a therapeutic molecule to treat a ciliopathy disorder the candidate molecule can include a small molecule, an antibody, a RNA interference molecule (RNAi), a short hairpin RNA (shRNA), or a small interfering RNA (siRNA).

The cell-based model can include a human retinal pigmentosa epicedium cell line (RPE). The ciliary gene can include the BBS4 gene. The human retinal pigmentosa epicedium cell line can stably expresses a short hairpin RNA (shRNA) against BBS4 expression. The defect can be hyper activation of the Wnt/3-cat signaling. In the RPE cell line based model, the candidate molecule can be a RNA interference molecule (RNAi), a short hairpin RNA (shRNA), or a small interfering RNA (siRNA), and determining the ability of the candidate molecule to rescue the defect can include transfecting the RPE cell line with the candidate molecule.

The animal-based model can include a morphant zebrafish embryo model, wherein the defect comprises a bbs4 depletion-induced convergent extension (CE) defect, a cerebellum organizational abnormality, and a renal development abnormality. In the morphant zebrafish embryo model, the candidate molecule can include a RNA interference molecule (RNAi), a short hairpin RNA (shRNA), or a small interfering RNA (siRNA), and the determining the ability of the candidate molecule to rescue the defect can include injecting the zebrafish embryo model with the candidate molecule.

The animal-based model can include an Ofd1 knockout mouse model.

One aspect of the present disclosure provides a method of treating a ciliopathic disorder in a subject comprising, consisting of, or consisting essentially of administering to the subject a therapeutically effective amount of a proteasome agonist such that the ciliopathic disorder is treated.

In some embodiments, the proteasome agonist comprises SFN. In other embodiments, the proteasome agonist comprises MVA.

### EXAMPLES

### Example 1

### Ciliopathy Proteins Reaulate Paracrine Sianalina by Modulating Proteasomal Degradation of Mediators

Cilia are critical mediators of paracrine signaling; however, it is unknown whether proteins that contribute to ciliopathies converge on multiple paracrine pathways through a common mechanism. It is shown in the presently provided disclosure that loss of cilopathy-associated proteins Bardet-Biedl syndrome 4 (BBS4) or oral-facial-digital syndrome 1 (OFD1) results in the accumulation of signaling mediators normally targeted for proteasomal degradation. In WT cells, several BBS proteins and OFD1 interacted with proteasomal subunits, and loss of either BBS4 or OFD1 led to depletion of multiple subunits from the centrosomal proteasome. In addition, it is demonstrated in the presently provided disclosure provided below that overexpression of proteasomal regulatory components or treatment with proteasomal activators sulforaphane (SFN) and mevalonolactone (MVA) ameliorated signaling defects in cells lacking BBS1, BBS4, and OFD1, in morphant zebrafish embryos, and in induced neurons from Ofd1-deficient mice. Furthermore, it is demonstrated in the presently provided disclosure that other proteasome-dependent pathways not known to be associated with ciliopathies are similarly defective in the absence of ciliopathy proteins. More specifically, it was found that loss of BBS1, BBS4, or OFD1 led to decreased NF-κB activity and concomitant IκBβ accumulation and that these defects were ameliorated with SFN treatment. Taken together, these data indicate that basal body proteasomal regulation can govern paracrine signaling pathways.

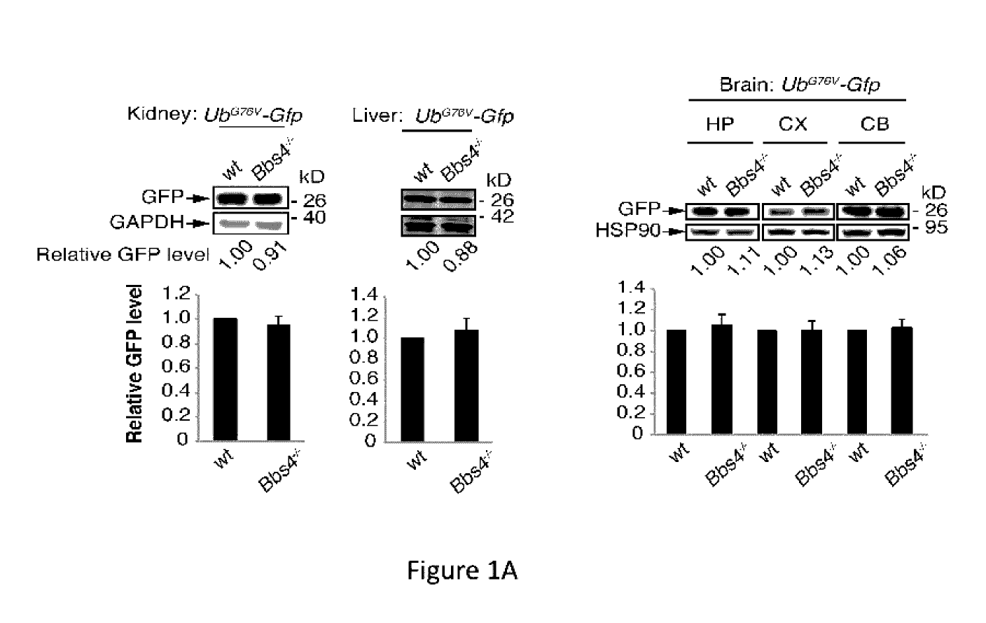

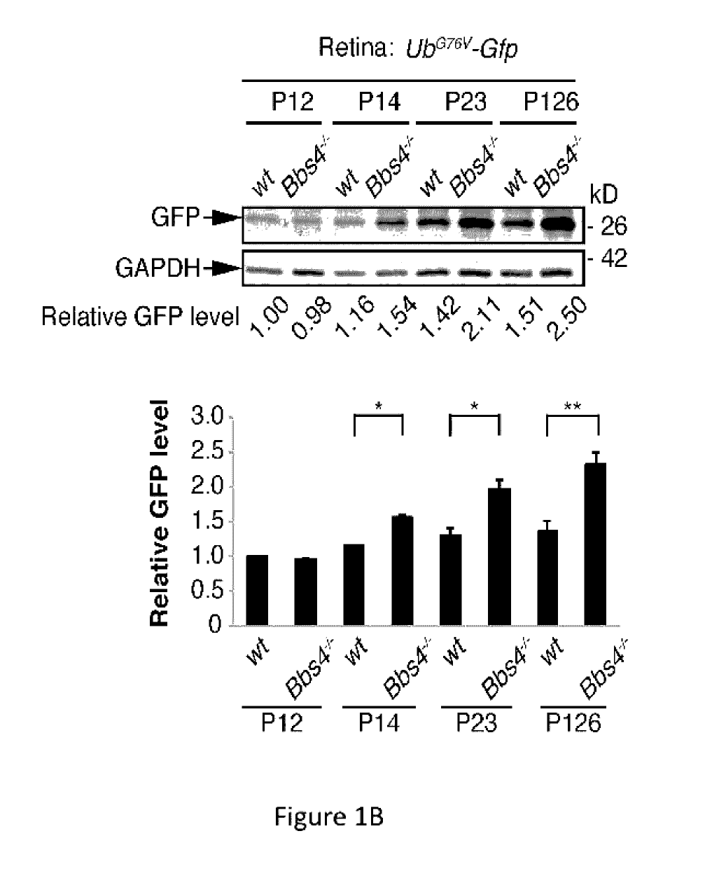

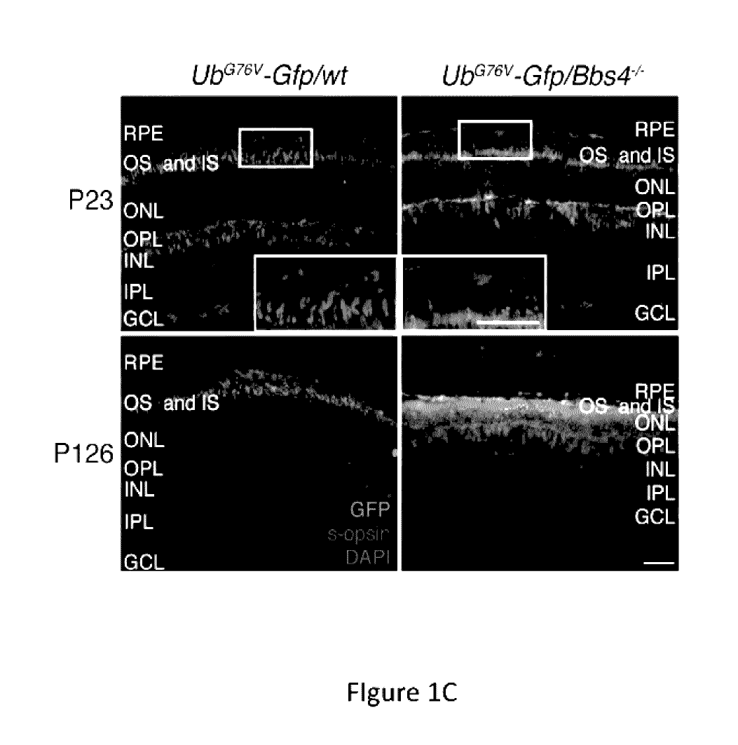

Accumulation of signaling mediators upon depletion of ciliopathy proteins. It has been reported previously that perturbation of some ciliopathy proteins alters the stability of Wnt signaling mediators such as β-catenin and Dishevelled (5, 8, 21, 29, 30). To study the physiological relevance of these findings in vivo, Bbs4^{-/-} mice were mated with a transgenic proteasome reporter mouse line expressing unstable ubiquitin-tagged Green Fluorescent Protein (GFP) (31) to generate Ub^{G76V}-Gfp Bbs4^{-/-} mice. As a hallmark of potential proteasomal dysfunction, GFP levels were examined in a broad range of tissues with known pathology in BBS and no aberrant GFP accumulation was observed in the kidney and liver of Ub^{G76V}-Gfp Bbs4^{-/-} mice (FIG. 1A) and modest increases were observed in components of the central nervous system (hippocampus, cortex, and cerebellum; FIG. 1A). By contrast, significant accumulation of GFP was detected from P14 onward in the retinas of Ub^{G76V}-Gfp Bbs4^{-/-} mice (FIG. 1B). To identify which cell types were accounting for the GFP signal, immunohistochemical analyses was performed of retina from three Ub^{G76V}-Gfp WT and three Ub^{G76V}-Gfp Bbs4^{-/-} mice (masked to the genotypes). Cryosections from Ub^{G76V}-Gfp Bbs4^{-/-} mice revealed significant aggregation of GFP in the rod and cone photoreceptor layers consisting of the outer segment, inner segment, and outer nuclear layer (FIG. 1C). This phenotype was restricted to photoreceptors (FIG. 1C) and was unlikely to be secondary to retinal degeneration, as no anatomical photoreceptor defects on P14 was observed, consistent with reported findings for other Bbs mouse mutants (29).

Given the specificity of GFP degradation defects in the photoreceptors of Bbs4^{-/-} mice and previous data on the abnormal stability of β-catenin in BBS4-depleted cells (5), the question was asked whether proteasomal dysfunction might be more broadly relevant to basal body-mediated paracrine signal regulation. If this is true, then: (a) other basal body proteins should have similar functions; and (b) other known basal body-mediated pathways should also be perturbed.

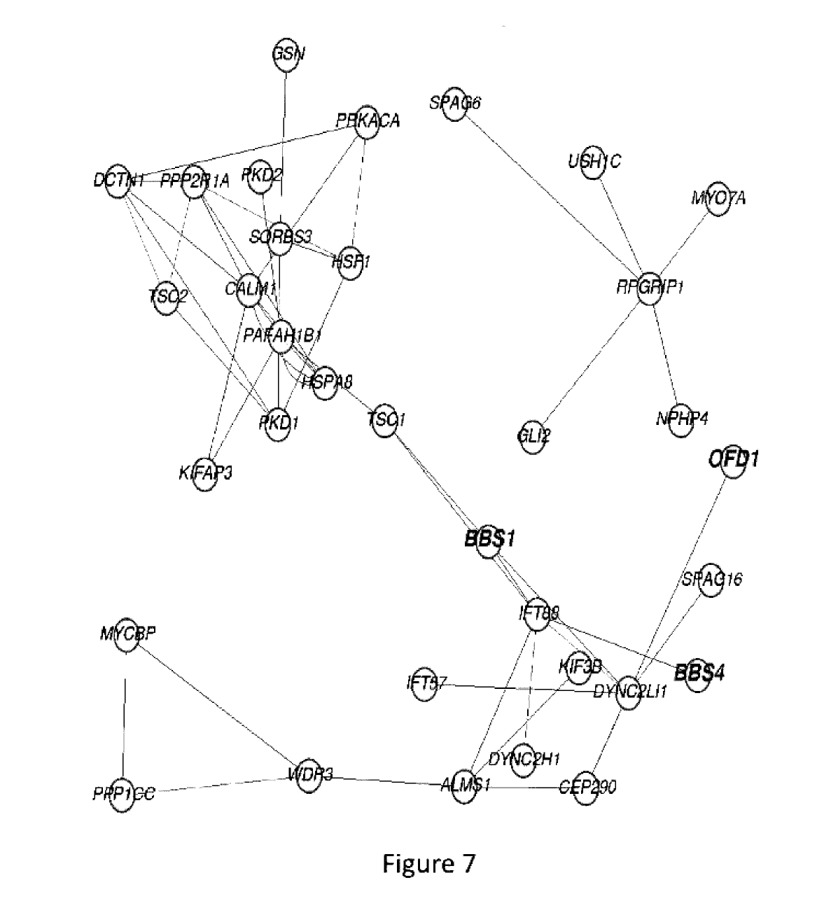

To evaluate functional links between basal body proteins, an in silico study was conducted ato analyze a network of coexpressed basal body and ciliary genes through a publicly available online tool (http://netview.tigem.it), which reconstructs the regulatory interactions among genes from genome scale measurements of gene expression profiles (32). This analysis revealed a subset of centrosomal transcripts (P=3.5×10⁻⁷) including BBS1, BBS4, and OFD1 (FIG. 7). The possibility was thus considered that loss of OFD1 can also lead to changes in the stability of signaling mediators.

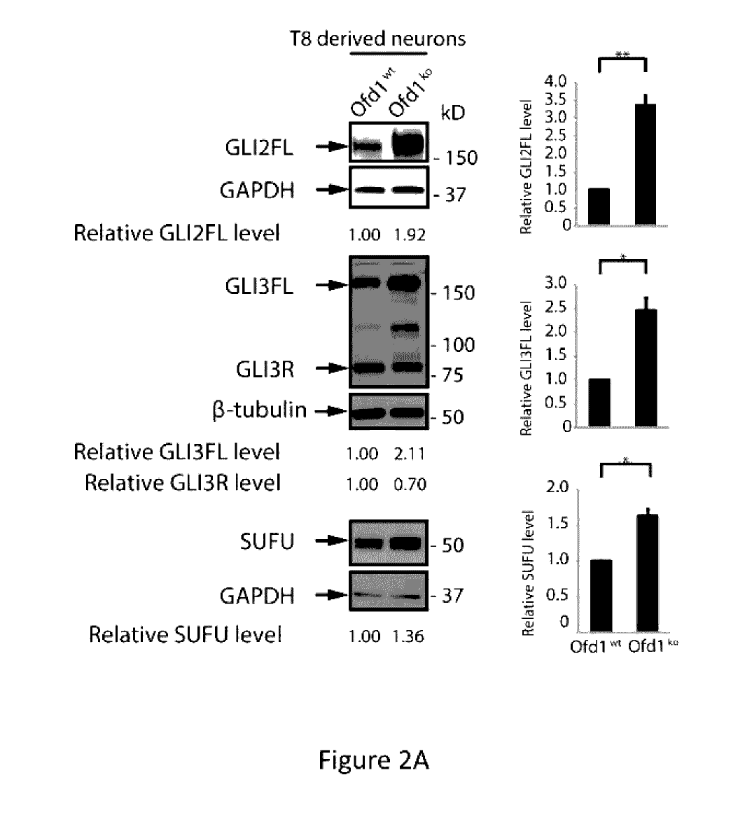

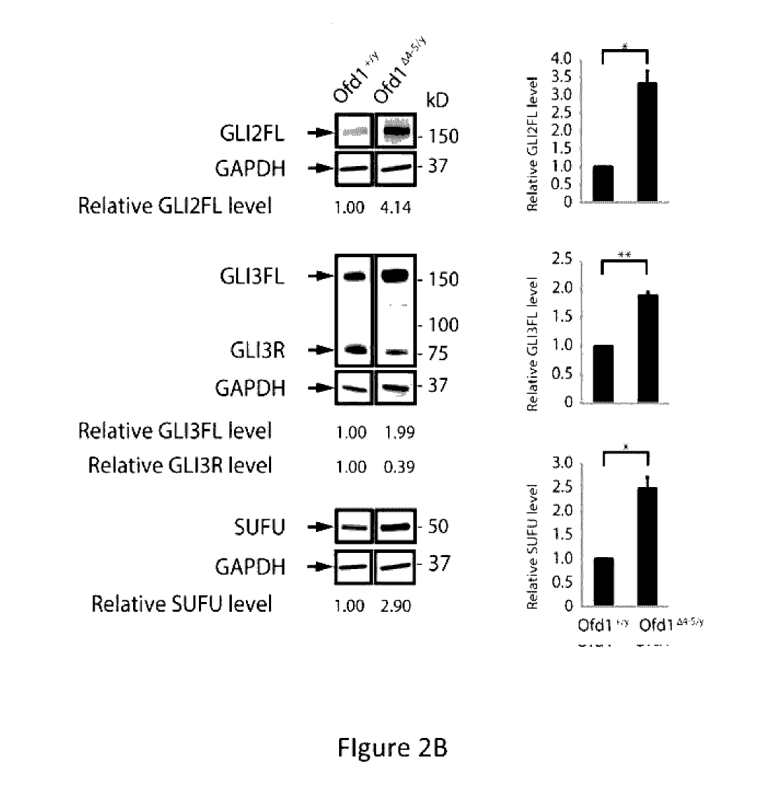

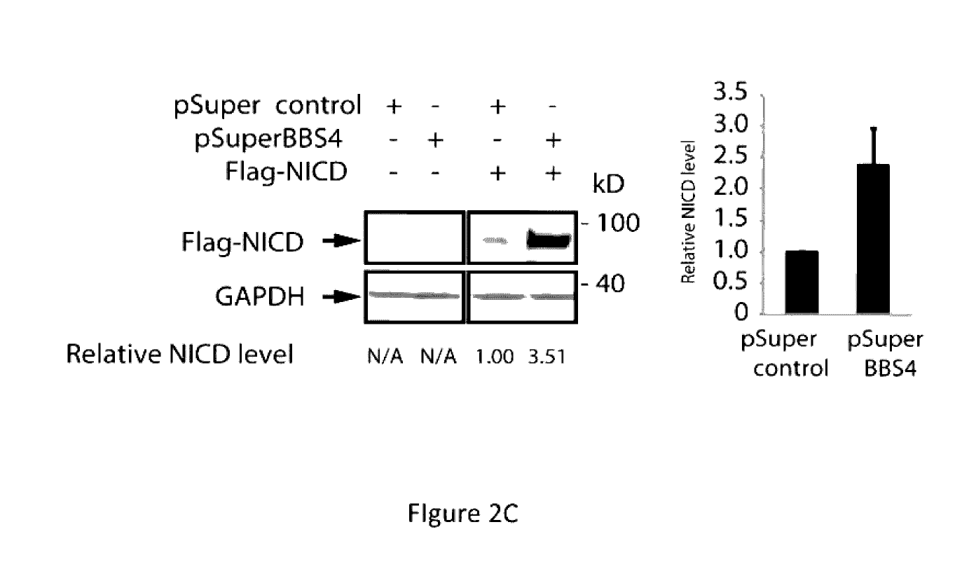

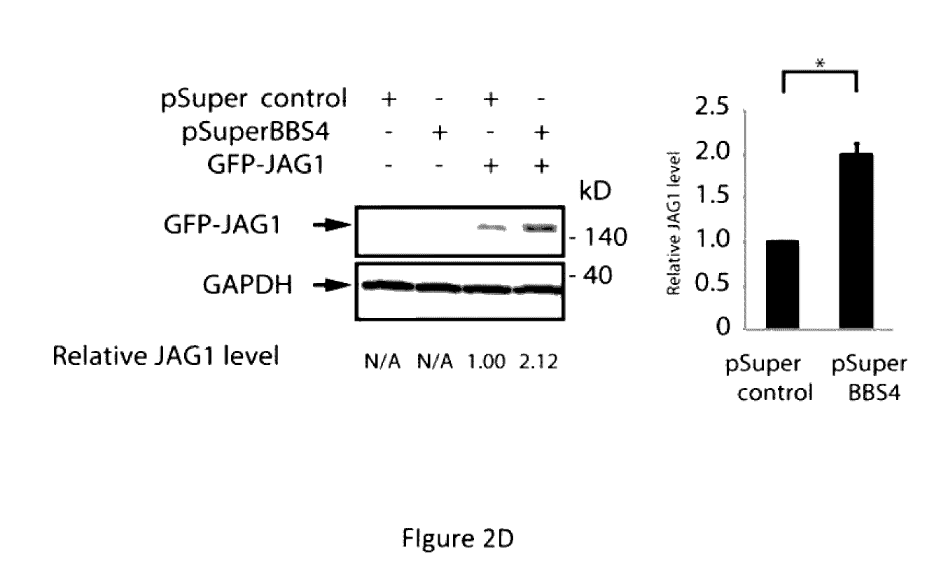

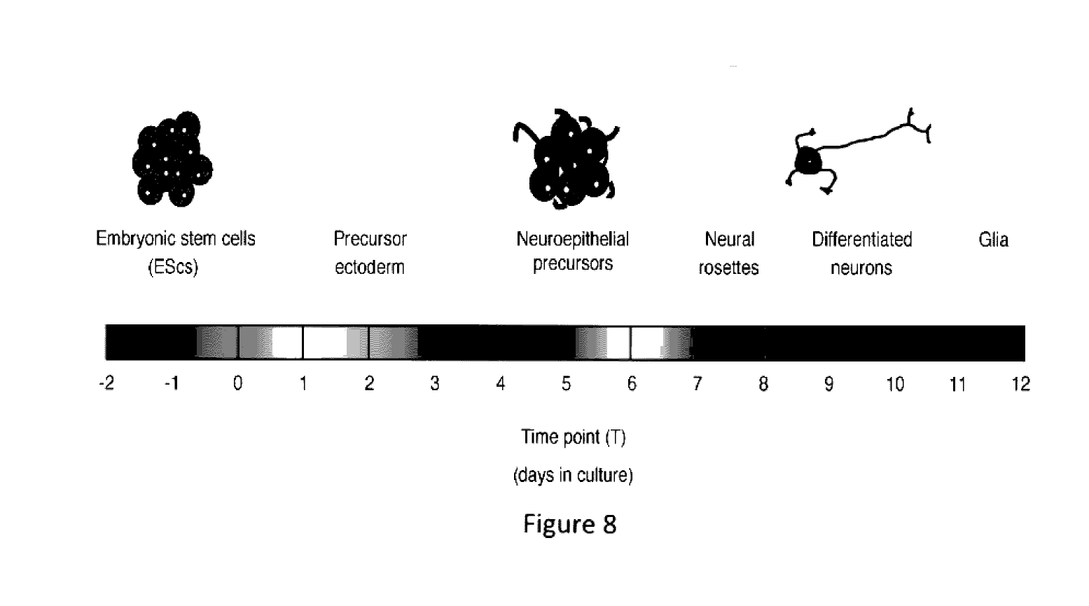

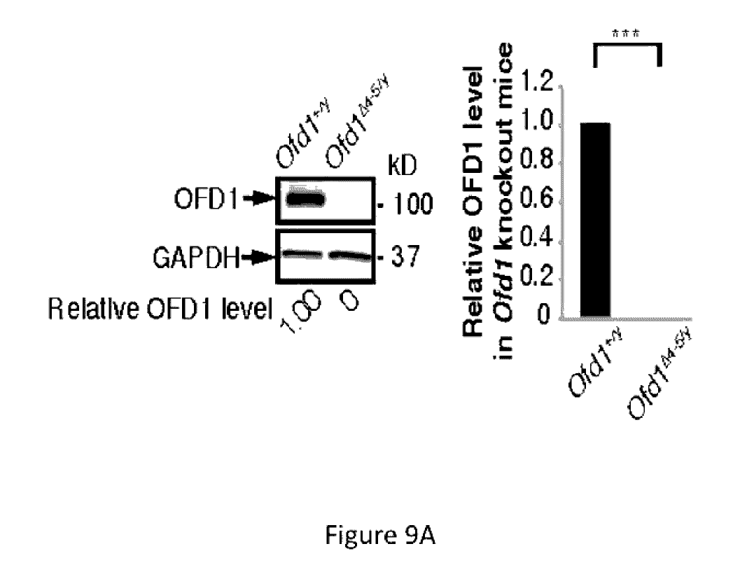

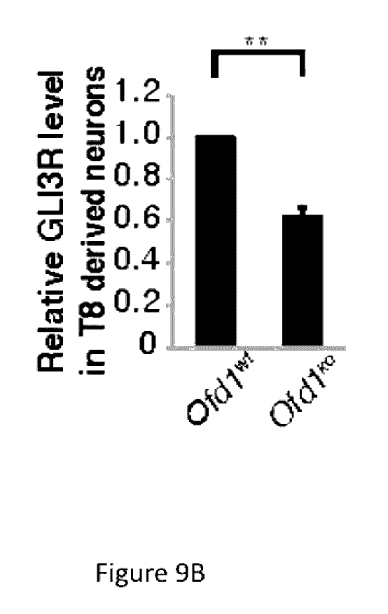

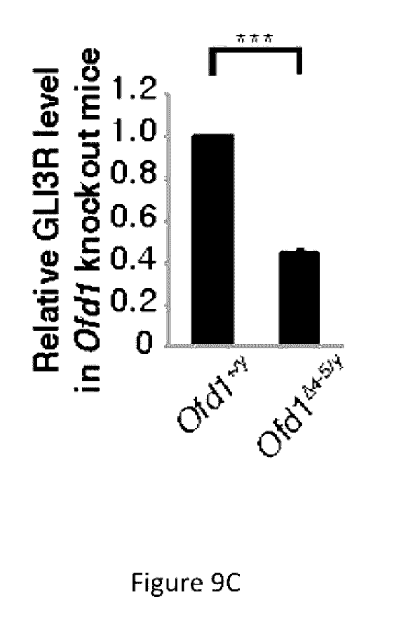

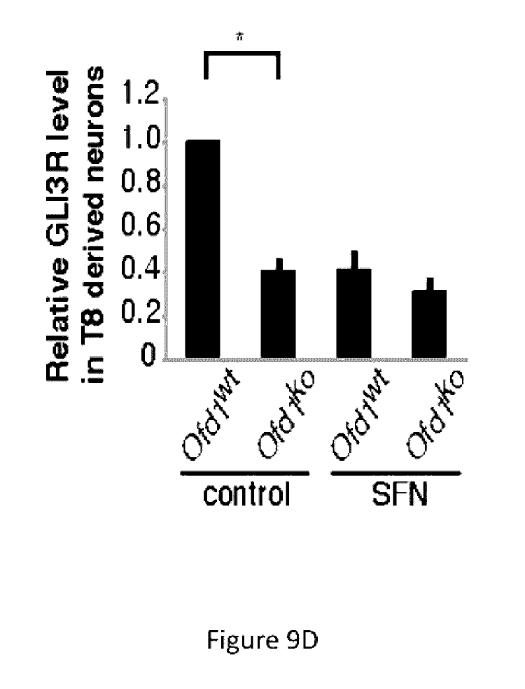

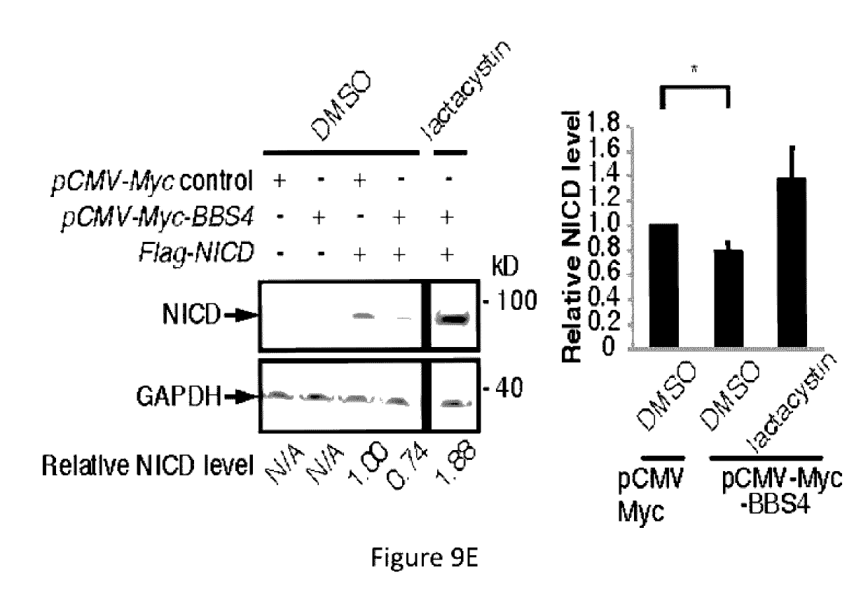

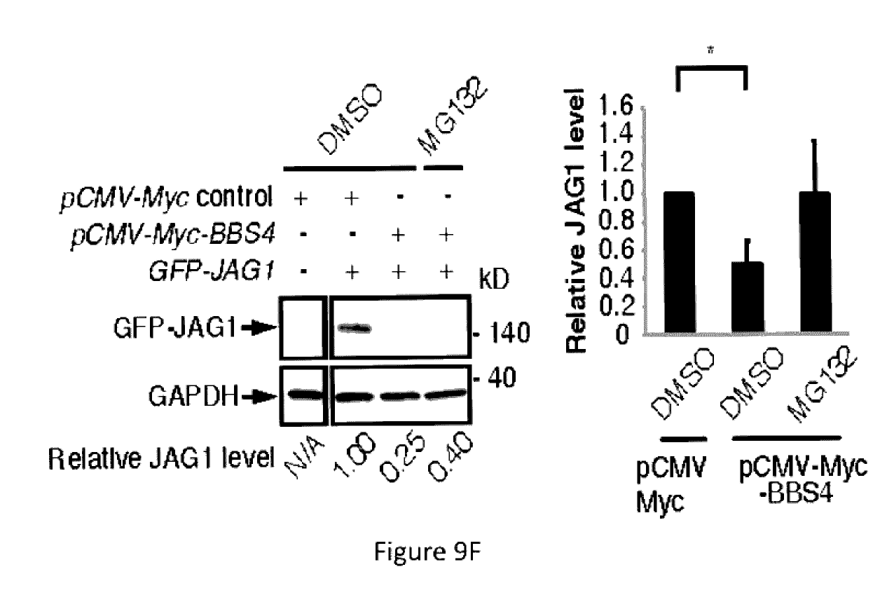

The established cilium-dependent paracrine signaling pathway, Hedgehog signaling (Hh), was used (2). In addition, an in vitro neural differentiation model was developed, in which the embryonic WT murine stem cells (WTESs) and Ofd1-deficient murine embryonic stem cells (KOESs) were cultured to generate neural progenitors (FIG. 8), a model that recapitulates the multi-step process of neural development in embryos (33). KOESs are male murine cells containing a gene trap mutation in Ofd1. As Ofd1 is to located on the X chromosome, Ofd1 KOESs are hemizygous for Ofd1 and do not produce the protein (34). Comparing Ofd1^{WT} neurons derived from WTESs and Ofd1^{KO} neurons from KOESs, a significant increase was found in GLI2FL (full-length GLI2) and SUFU at the T8 time point in Ofd1-depleted neurons (FIG. 2A). Increased levels of GLI2FL and SUFU were also found in lysates from Ofd1^{Δ4-5/+} heterozygous female mouse embryos (data not shown) and Ofd1^{Δ4-5/y} hemizygous male mouse embryos (FIG. 9A) compared with levels observed in WT littermates (FIG. 2B). In addition, GLI3FL (full-length GLI3) increased both in Ofd1^{KO} neurons (FIG. 2A) and in Ofd1^{Δ4-5/+} (data not shown) and Ofd1^{Δ4-5/y} mutant embryos (FIG. 2B), with a concomitant decrease in glioma-associated oncogene 3 repressor (GLI3R) levels (FIGS. 2, A and B, and FIGS. 9, B and C).

Similar to Hh and Wnt signaling, Notch signaling is regulated through the cilium (13), and if the overarching hypothesis is correct, this pathway should also be perturbed in the mutants produced as described herein. As a first test, Flag-tagged Notch1 intracellular domain (NICD) was cotransfected into HEK-293-FT cells depleted of BBS4 by shRNAmediated gene silencing (pSuperBBS4) (5). Immunoblot analysis of transfected cells revealed an elevation of NICD protein levels upon knockdown of BBS4 (FIG. 2C). Further, the question was asked whether loss of BBS4 modulates the protein levels of a DSL ligand, Jagged 1 (JAG1). Therefore, GFP-tagged JAG1 was assayed in BBS4-depleted and control cells. Similar to findings with NICD, knockdown of BBS4 also resulted in an elevation of GFP-JAG1 (FIG. 2D).

Selective Disruption of UPS Caused by Loss of Ciliopathy Proteins.

Given the data on Wnt, Shh, and Notch and the fact that the processing of components from these paracrine signaling pathways is known to be mediated by the proteasome (11, 35), the question was asked whether proteasomal agonists could ameliorate the signaling phenotypes of basal body mutants.

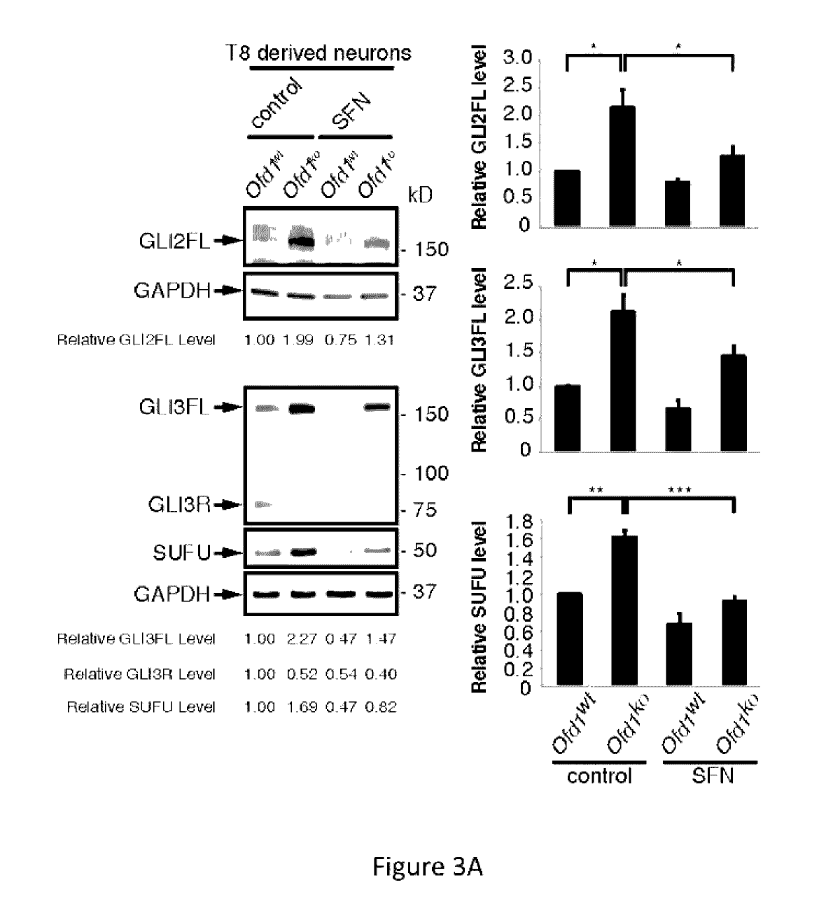

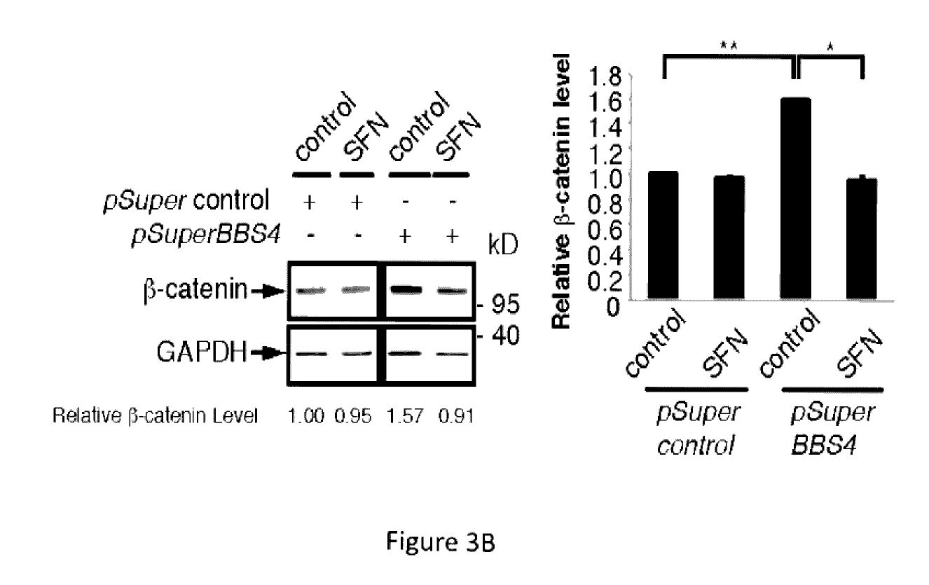

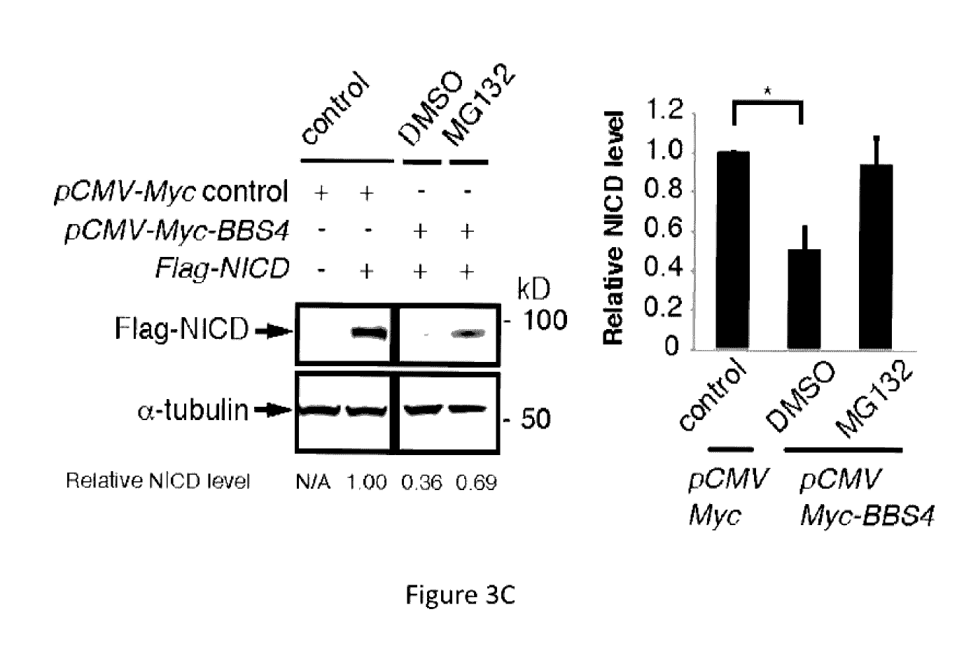

The expression levels of three catalytic subunits of the proteasome, proteasome subunit 13 types 5, 6, and 7 (PSMB5, PSMB6, and PSMB7), as well as the corresponding peptidase activities, increase upon treatment with sulforaphane (36) [SFN; 1-isothiocyanato-4(R)-methylsulfinylbutane], an isothiocyanate extracted from cruciferous vegetables, rendering this compound an attractive initial substrate for the studies. It was found that treatment with SFN rescued the accumulation of GLI2FL, upon overexpressing the proteins of interest. Therefore, BBS4 was overexpressed in cells and a depletion was observed of both NICD (FIG. 3C and FIG. 9E) and JAG1 (FIG. 9F); further, BBS4-overexpressing cells were treated with two proteasome inhibitors, Ncarbobenzoxyl [symbol] leucinyl [symbol] leucinyl [symbol] norleucinal (MG132) and lactacystin as well as DMSO vehicle as a control. While cells overexpressing BBS4 showed a 21%-50% reduction in total NICD levels (FIG. 3C and FIG. 9E) and an approximately 50%±16% reduction in total JAG1 levels (FIG. 9F), treatment with MG132 and lactacystin restored NICD protein levels to 93% (FIG. 3C) and 138% (FIG. 9E) of basal levels, respectively, and restored JAG1 levels to 99%±38% of basal levels (FIG. 9F), suggesting that BBS4 can facilitate the degradation of Notch signaling mediators in a proteasome-dependent manner.

Regulation of the Proteasome Complex by Ciliopathy Proteins.

Although the presently disclosed observations in mouse mutants, zebrafish embryos (5), and cultured cells argue that these basal body proteins are responsible for regulating the proteasome, the mechanism of that action is unclear, leaving the possibility that the observed phenotypes are surrogate effects of generalized GLI3FL, and SUFU in T8-derived Ofd1^{KO} neurons (FIG. 3A). Consistent with the participation of the proteasome in both the processing and degradation of GLI2/3, treatment with SFN reduced GLI3R protein levels (FIG. 3A and FIG. 9D). Moreover, SFN treatment of cells depleted of BBS4 also revealed a reduction in β-catenin protein levels relative to vehicle-treated to cells (FIG. 3B), suggesting that defects in proteasome-dependent processing and degradation of GLI2/3, SUFU, and β-catenin can be ameliorated.

Taken together, these data argue for a role of basal body proteins in regulating proteasome-mediated degradation and that a reciprocal signaling/proteasomal phenotype cellular “malaise” should be observed. Therefore the possibility was explored of direct biochemical relationships between BBS1, BBS4, and OFD1 and the proteasome.

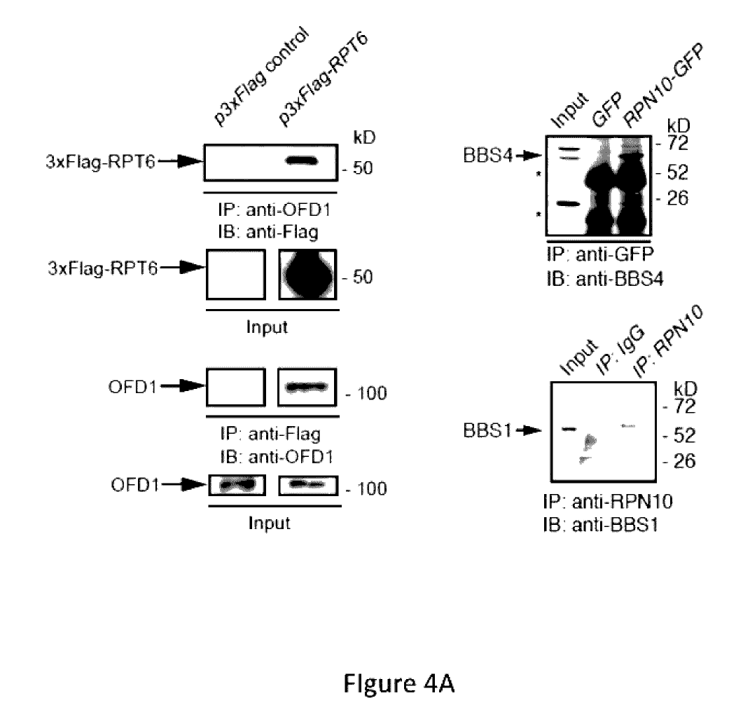

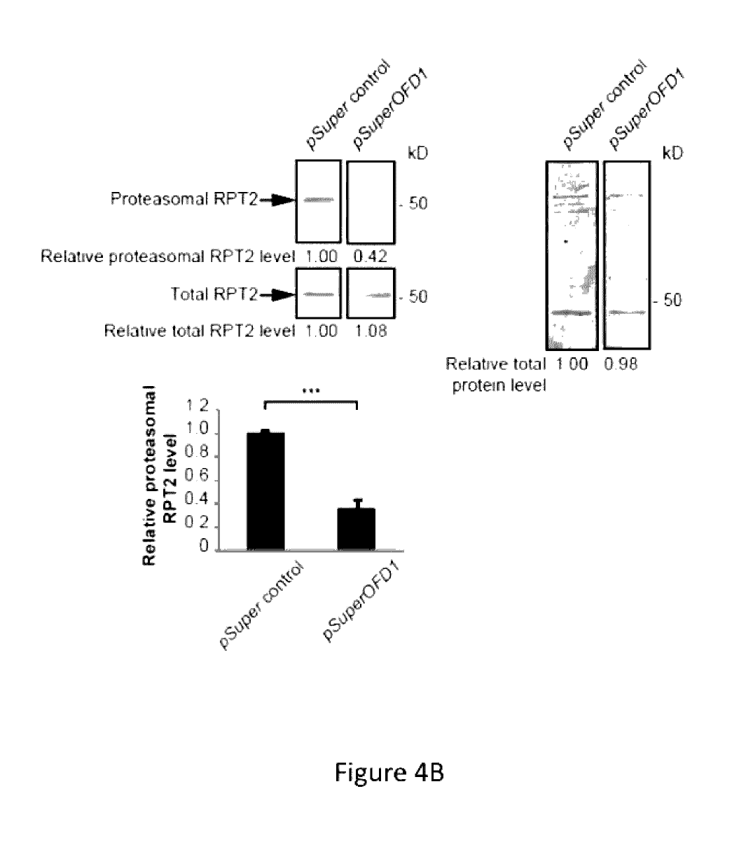

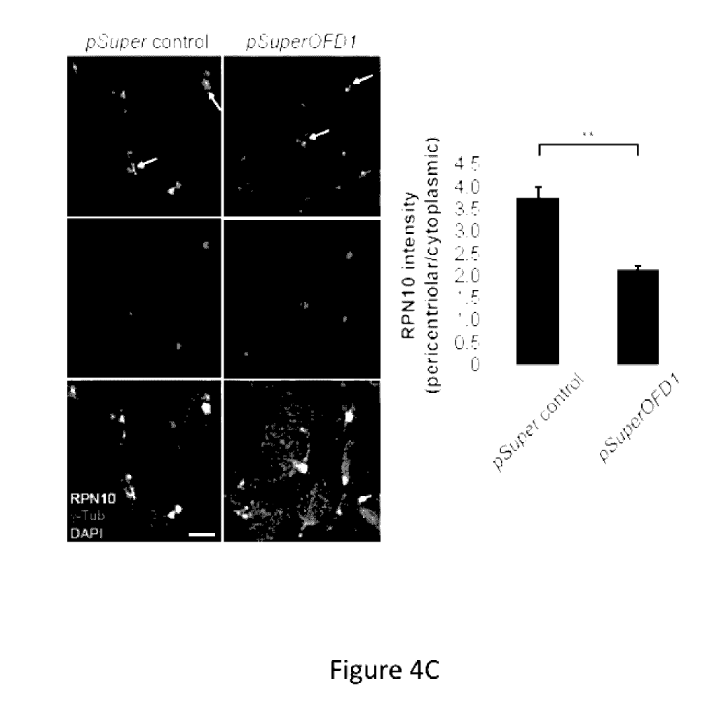

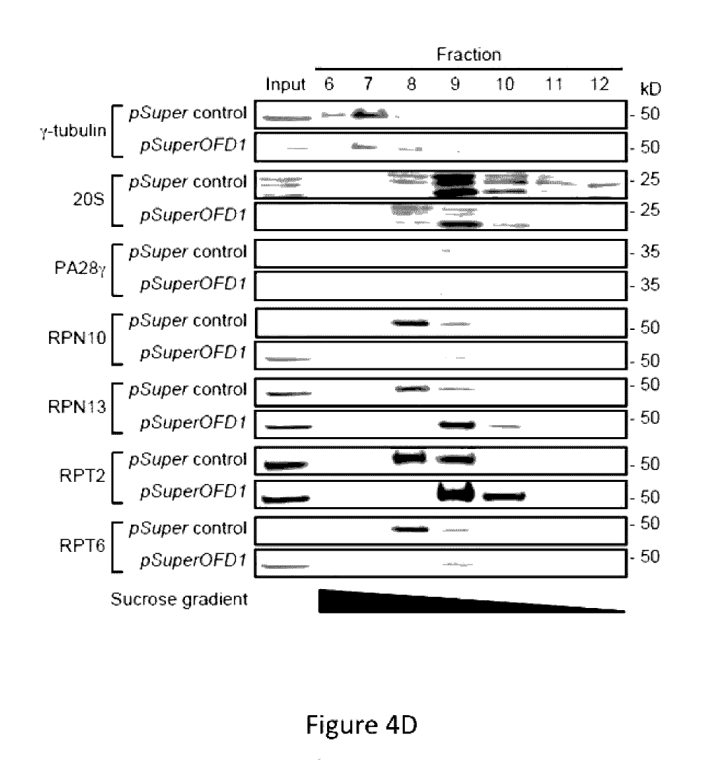

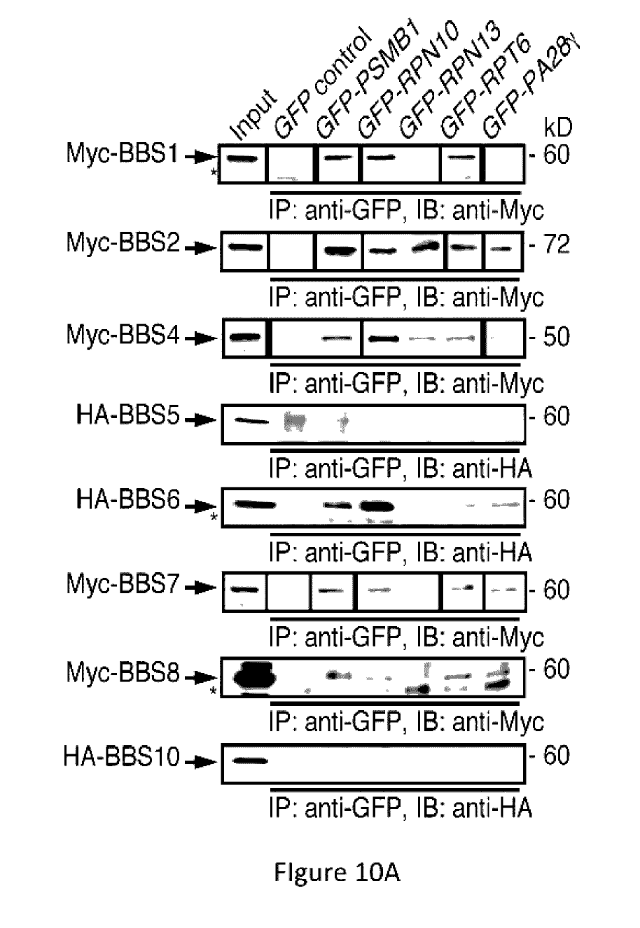

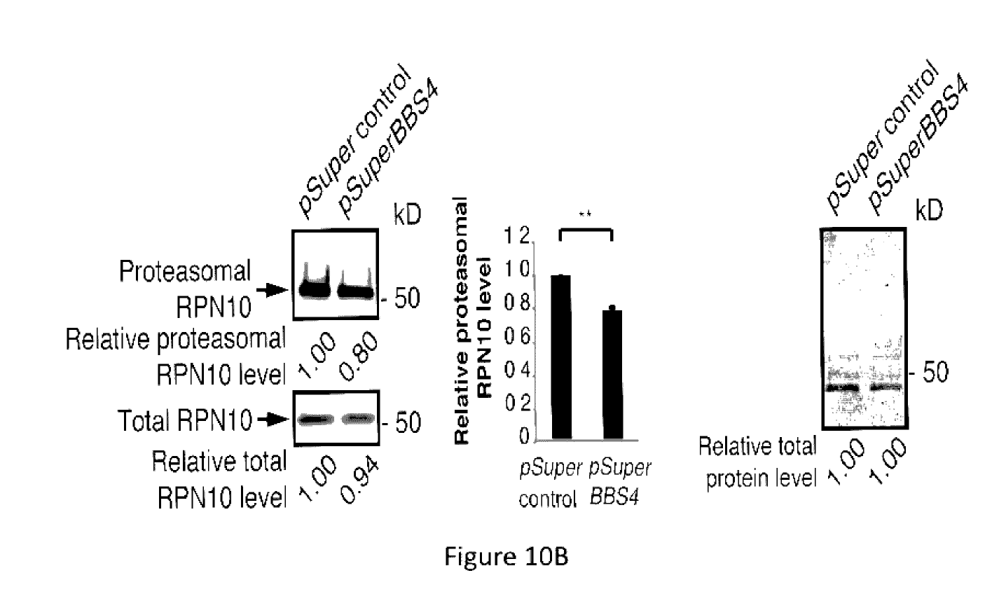

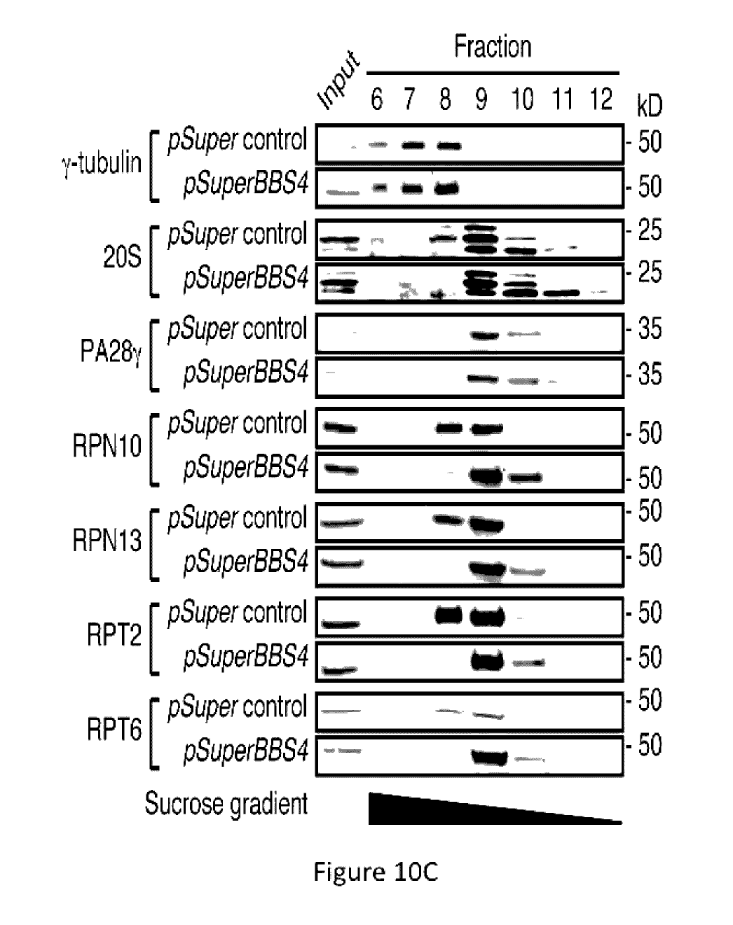

First, an unbiased mass spectrometry screen was conducted to identify putative OFD1-interacting proteins. This experiment uncovered a spectrum of proteasomal subunits including regulatory proteasome ATPase subunit 6 (RPT6), a finding confirmed by semiendogenous coimmunoprecipitation (FIG. 4A). Independently, protein-protein interactions were tested between multiple BBS proteins (BBS1, 2, 4, 5, 6, 7, 8, and 10) and several proteasomal subunits (proteasome subunit [type 1 subunit in the 20S particle [PSMB1], regulatory proteasome non-ATPase subunit 10 [RPN10], regulatory proteasome non-ATPase subunit 13 [RPN13], RPT6, and non-19S regulatory subunit [PA28γ]. Under stringent detergent conditions (1% Triton X-100), the previously reported interaction was confirmed between BBS4 and RPN10 (5) by semiendogenous coimmunoprecipitation (FIG. 4A) and a biochemical interaction was also observed of some BBS proteins (BBS1, BBS2, BBS4, BBS6, BBS7, and BBS8) with proteasomal components, while BBS5 and BBS10 did not interact with any tested proteasomal subunits (FIG. 10A). To test the physiological relevance of a BBS-proteasome interactome, it was determined that BBS1 and RPN10 interacted at endogenous levels in protein lysates isolated from C57BL/6 mouse testes (FIG. 4A). To probe the relevance of these interactions, the question was asked whether loss of OFD1 or BBS4 alters the composition of the proteasome with respect to its subunits. Using a stable cell line expressing tagged regulatory proteasome non-ATPase subunit 11 (RPN11) (37), OFD1 was suppressed, 26S proteasome complexes were purified, and a robust reduction was observed (60%±8%) of 26S-bound regulatory proteasome ATPase subunit 2 (RPT2) in OFD1-depleted cells in comparison with that in control, while total RPT2 protein abundance was not affected by OFD1 depletion (FIG. 4B).

The fraction of 26S-bound RPN10 in BBS4-depleted cells was also assayed and a modest but significant reduction (20%±3%) in RPN10 was observed (FIG. 10B). Furthermore, the localization of RPN10 around the centrosome in HEK-293 cells depleted of BBS4 and OFD1 was also tested by quantifying the RPN10 signal that localized around centrosomal γ-tubulin; appreciable changes were not observed in BBS4-depleted cells (data not shown), possibly because of a lack of spatial resolution. By contrast, a 43%±2% reduction of pericentriolar RPN10 levels was observed (normalized to cytoplasmic RPN10 levels) in OFD1-depleted cells (FIG. 4C). Given these data, whether loss of OFD1 or BBS4 altered the composition of RPN10, RPT2, and other proteasomal components was tested by sedimentation. Based on the commercial availability of reliable antibodies against proteasomal subunits, it was found that RPN10, RPN13, RPT2, and RPT6 were enriched in γ-tubulin-enriched fractions in control cells (FIG. 4D and FIG. 10C). Depletion of OFD1 or BBS4, however, resulted in a shift in the peak expression of the proteasomal subunits (FIG. 4D and FIG. 10C). Taken together, these data suggest that BBS proteins and OFD1 regulate the composition of the centrosomal proteasome, likely through direct biochemical interactions.

Activation of Proteasomal Components Ameliorates Signaling Defects Caused by Loss of Ciliopathy Proteins that Reside at the Basal Body.

These observations next led to speculation that (a) if reduction of proteasomal subunits in the centrosomal fraction of BBS4 or OFD1-depleted cells results in hampered degradation of signaling mediators, then increasing expression of the corresponding proteasomal subunits may ameliorate defective signaling phenotypes; and (b) activation of the proteasome might compensate for signaling defects.

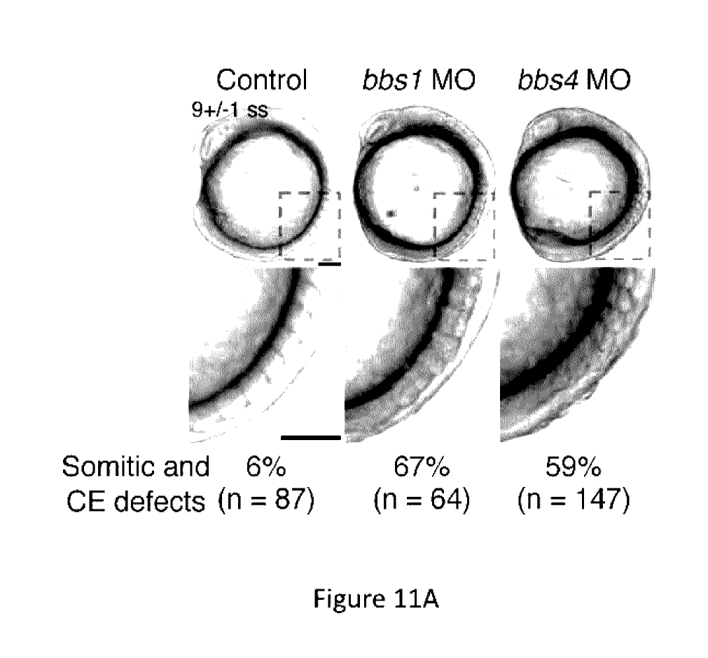

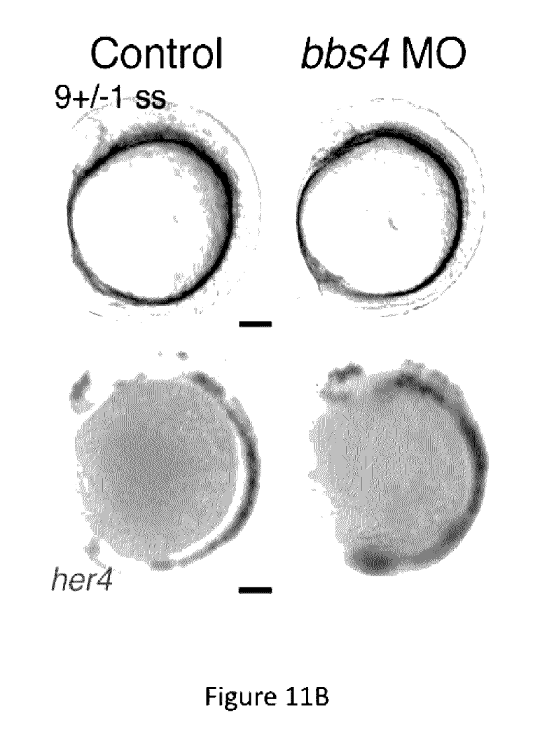

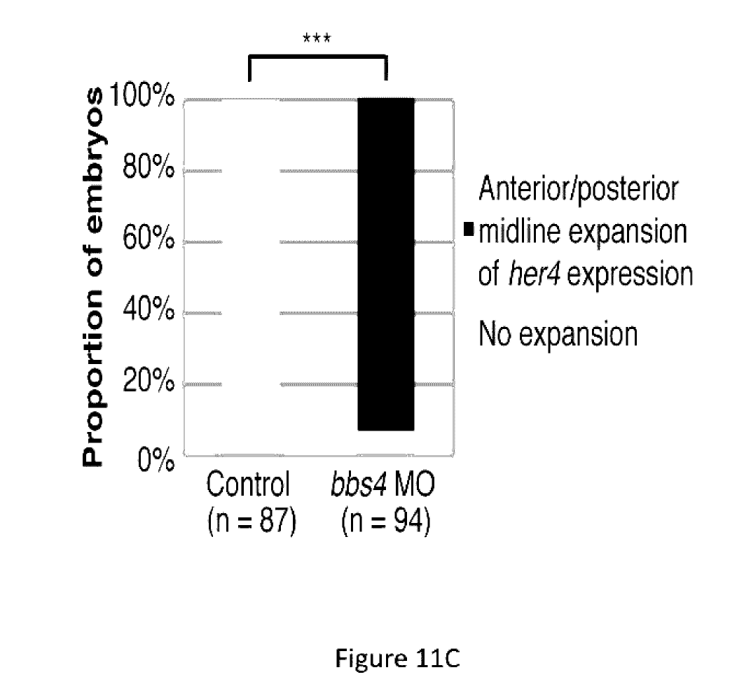

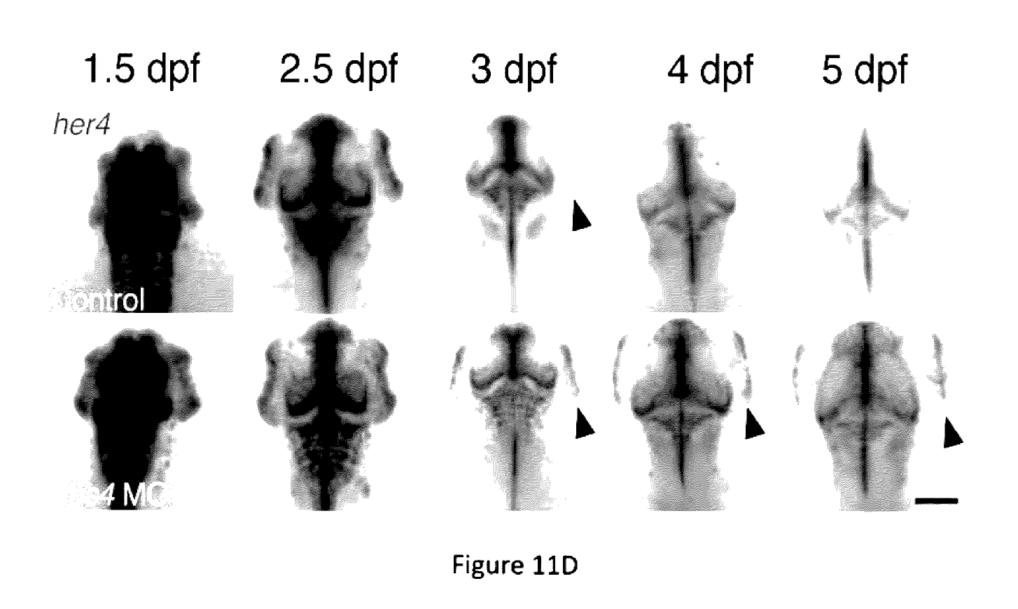

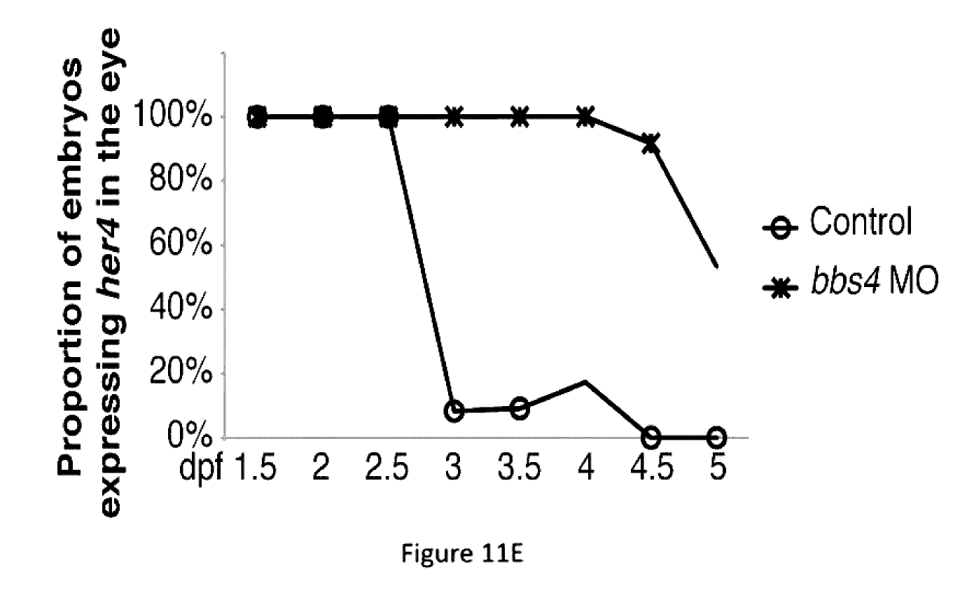

It has been reported previously that loss of ciliopathy proteins produces defects in convergent extension (CE) movements during gastrulation in zebrafish embryos (5, 29, 30). In addition to and independent of these phenotypes, defects were observed in the present disclosure in the definition of somite boundaries in zebrafish embryos injected with morpholinos against bbs genes (bbs morphants [bbs MO]) (FIG. 11A and ref. 38). In vertebrates, somites develop as epithelial blocks at a temporal and spatial periodicity that is controlled, in part, by Notch signaling (39). Given the phenotypic overlap between bbs morphants and segmentation mutants (mib, bea, des, and ael) (39), as well as the instant biochemical Notch observations in BBS4-deficient cultured mammalian cells, the question was asked whether dysfunction of these proteins might influence Notch signaling.

Upon injection of the established bbs4 morpholino (5, 38) into 1 to 8-cell-stage embryos, and scoring at the 9±1 somite stage (9±1 ss), 92% of embryos exhibited an expansion of the anteroposterior midline expression domain of her4 (FIG. 11B), whose transcription is targeted directly by notch1 activation (40). To differentiate between ectopic expression at developmental stages, when Notch signaling is already active, and the possibility that signaling is persistent throughout stages when it should be diminished, an incremental developmental series of her4 in situ hybridization over the first 5 days of development was performed. Scoring WT embryos (staged by number of somites and, later, by the presence of anatomical features such as the swim bladder to ensure that embryos of the same age were compared across experiments), it was observed that the expression of her4 in neural structures of the head, including the developing forebrain, midbrain, hindbrain, and eye, was robust through 2.5 days post fertilization (dpf) and then began to wane. In bbs4 morphants, persistent her4 expression was observed through 5 dpf, especially in the eye (FIG. 11C).

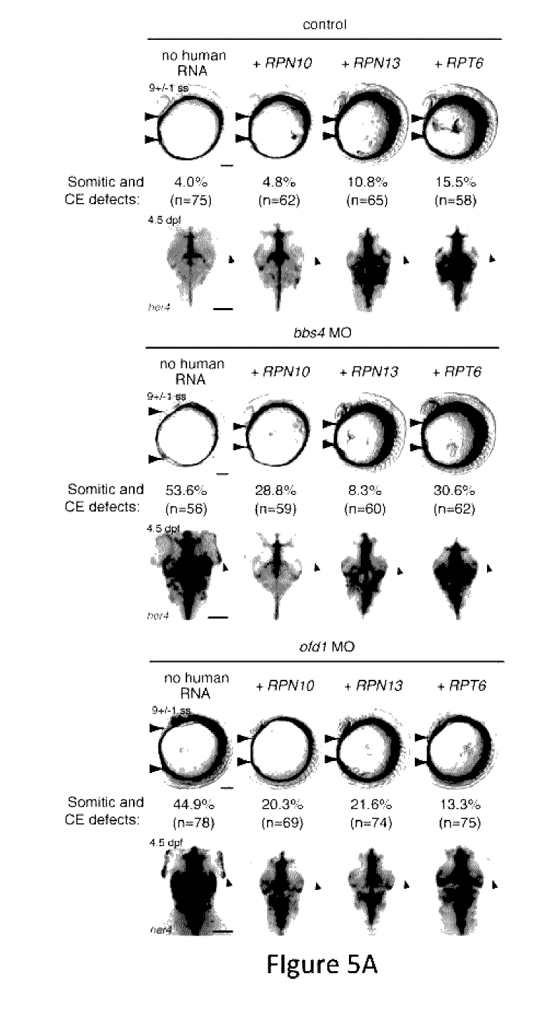

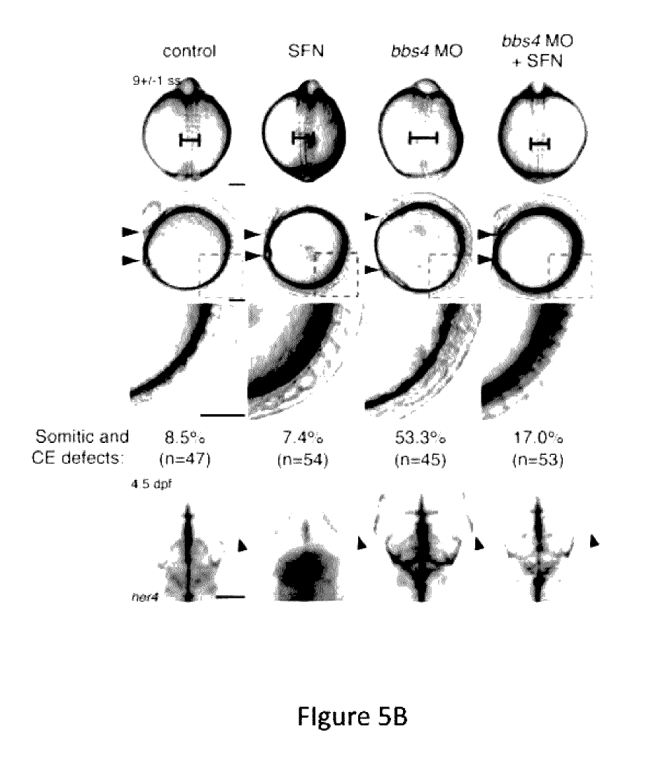

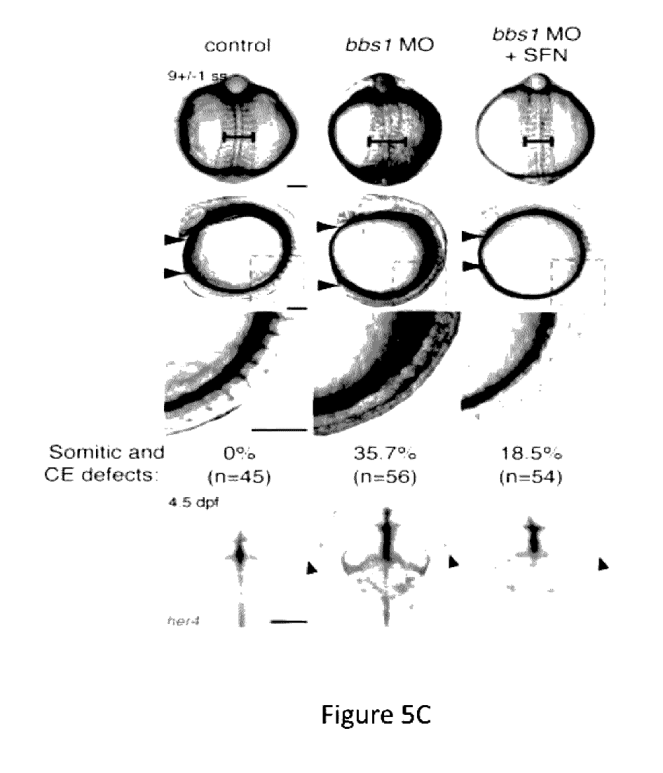

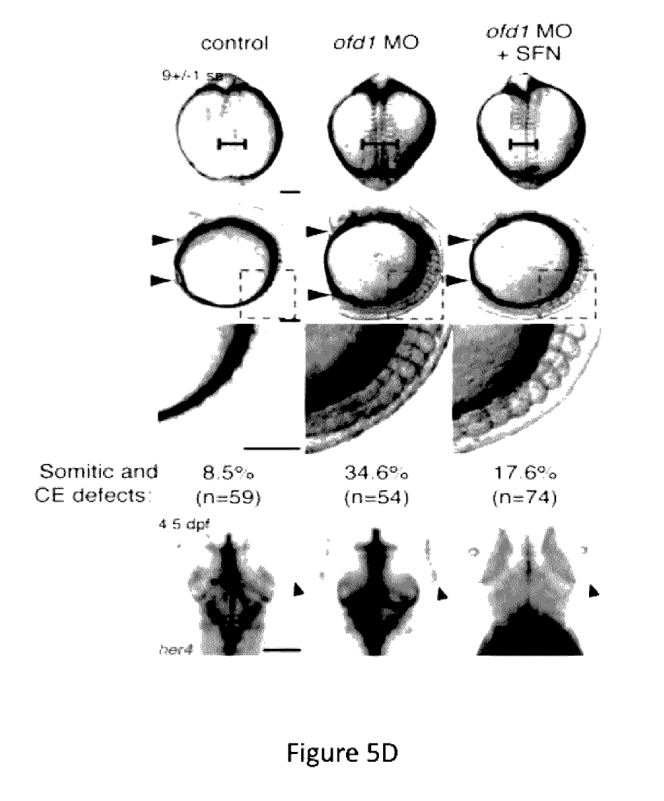

Given the availability of experimentally tractable signaling phenotypes, the question was asked whether overexpression of proteasomal subunits that were seen to mislocalize in BBS4 and OFD1-depleted cells might rescue the bbs4 and ofd1 morphant phenotype. Upon blind scoring for the effects of coinjecting human RPN10, RPN13, or RPT6 mRNA with the bbs4 or ofd1 morpholino, substantial rescue of defective CE, somitic definition, and persistent her4 expression was measured (FIG. 5A).

Next, as an independent test, bbs4 morphants coinjected with SFN, a known transcriptional activator of proteasomal subunits (36) were examined. Upon blind scoring at the 9±1 ss, there was a significant reduction of bbs4 morphants with CE and somitic defects (P<0.001; FIG. 5B). Moreover, coinjection of SFN with a bbs4 morpholino ameliorated the ectopic expression of her4 in neural structures at 4.5 dpf (FIG. 5B). A similar rescue of bbs1 and ofd1 morphants was also observed, in which coinjection of SFN gave rise to the same robust rescue of defective CE phenotypes, somitic defects, and persistent her4 expression as that observed in bbs4 loss-of-function models (FIGS. 5, C and D).

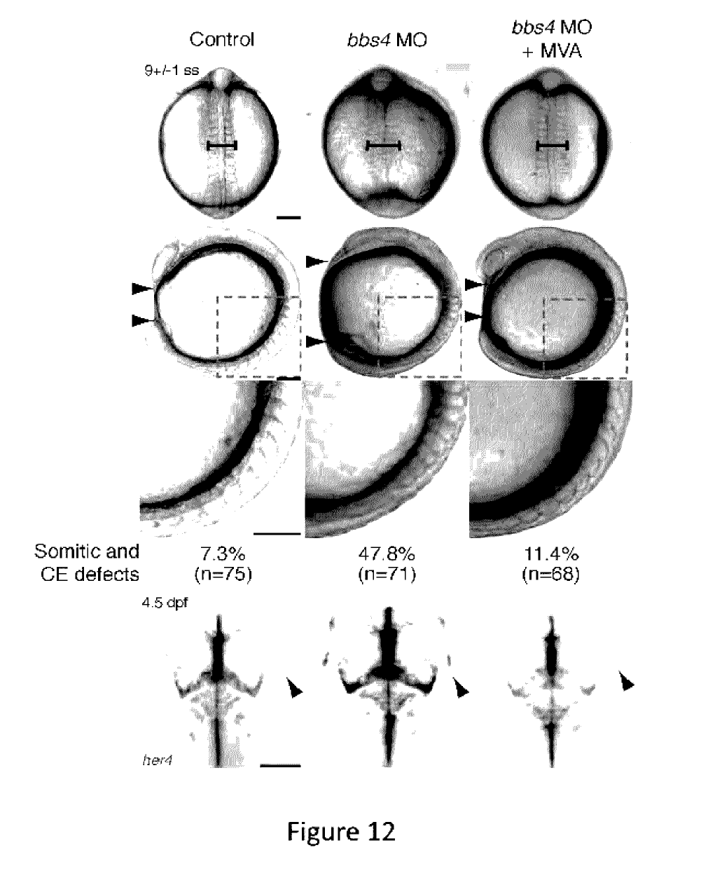

To substantiate the rescue effects of SFN, another proteasomal agonist was used, mevalonolactone [known as mevalonic acid lactone, mevalonate, and (±)-β-hydroxy-β-methyl-δ-valerolactone and abbreviated hereafter as MVA], to ask whether broad activation of the proteasome can alleviate basal body-dependent phenotypes. MVA was coinjected with the bbs4 morpholino into zebrafish embryos, and upon blind scoring at 9±1 ss, a reduction in bbs4 morphant zebrafish with CE defects from 47.8% to 11.4% was found with MVA (FIG. 12). To investigate the persistence of Notch signaling in bbs4 morphant zebrafish coinjected with MVA, in situ hybridization for her4 was performed. Coinjection of MVA with a bbs4 morpholino reduced her4 expression levels in neural structures, especially the retina, to those in WT zebrafish by 4.5 dpf (FIG. 12).

NF-κB Signaling Defects in Basal Body Ciliopathy Mutants can be Rescued by Activation of the Proteasome.

The presently disclosed findings indicate that Wnt, Notch, and Shh phenotypes generated upon loss of three basal body proteins might converge at the to point of proteasomal degradation. Therefore a model was considered in which the basal body region serves as a broad regulator of paracrine signaling through proteasomal degradation. If this model is true, then (a) other paracrine pathways not implicated previously in ciliary/basal body biology, but known to be regulated by the proteasome, should exhibit signaling defects upon depletion of BBS proteins or OFD1; and (b) the model should also be able to predict the direction of the phenotype (suppressed or excessive signaling). To test this, NF-κB signaling was used, a pathway involved in inflammatory responses and lymphoid organogenesis with no known link to basal body (dys)function. In response to stimuli, IκBs are degraded by the proteasome, releasing NF-κB to translocate to the nucleus and activate the transcription of target genes (41).

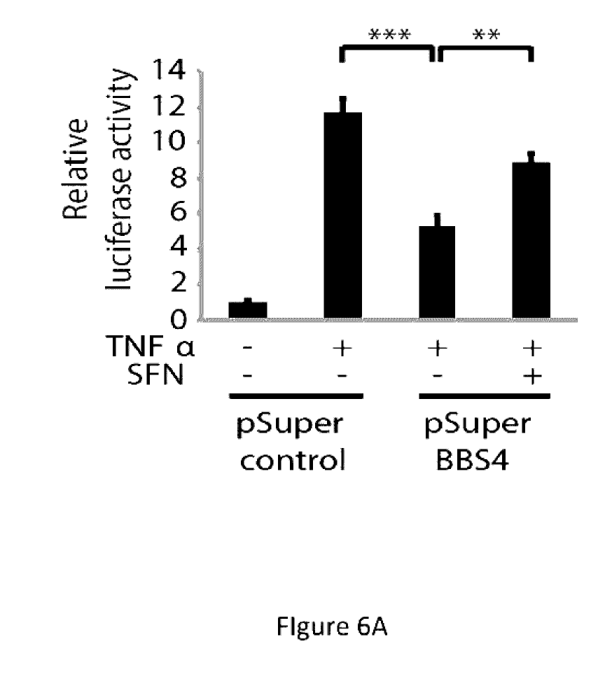

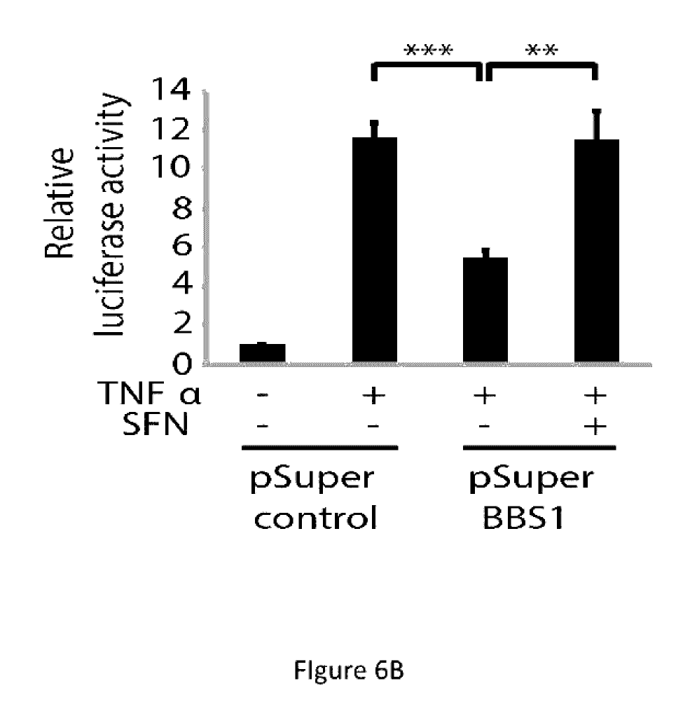

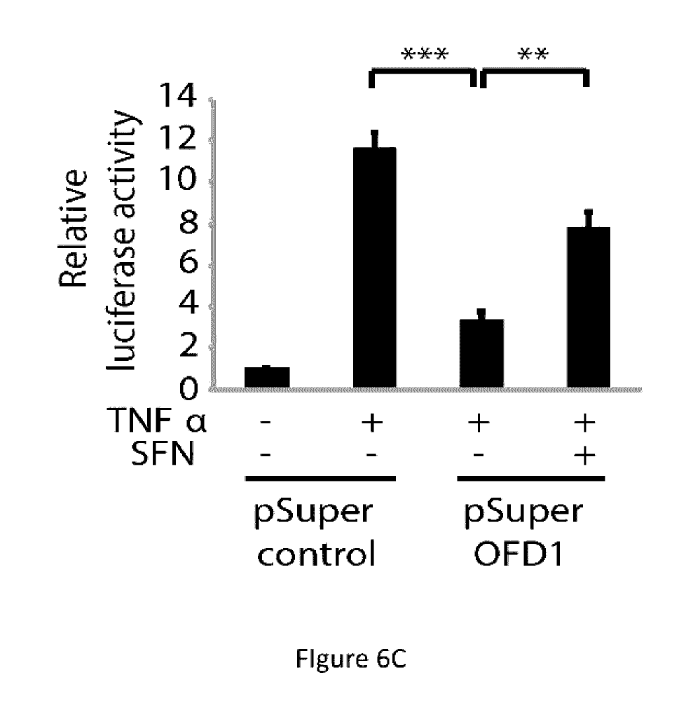

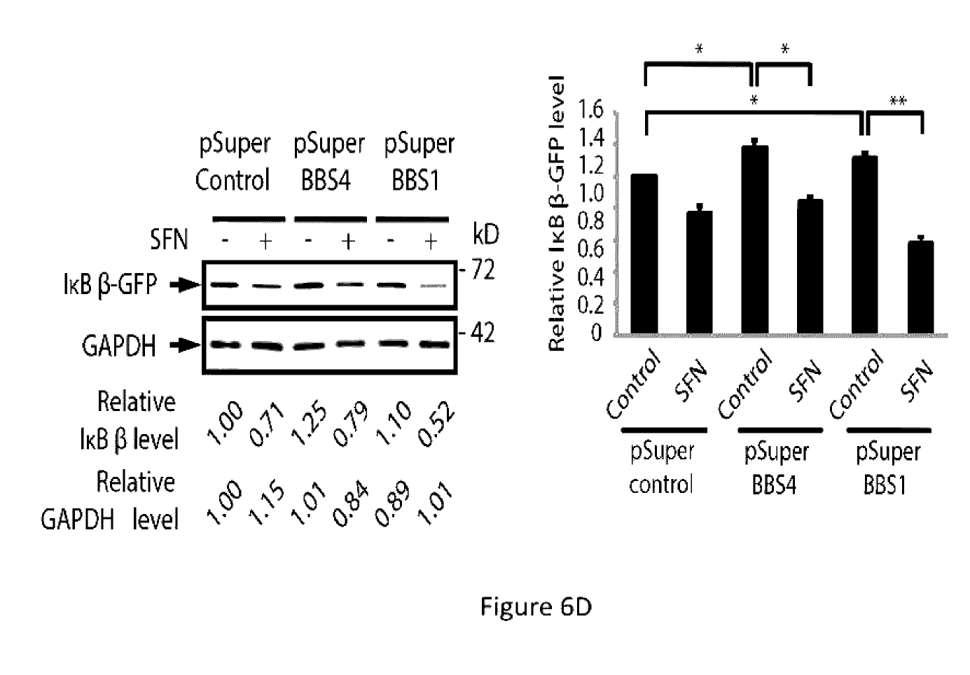

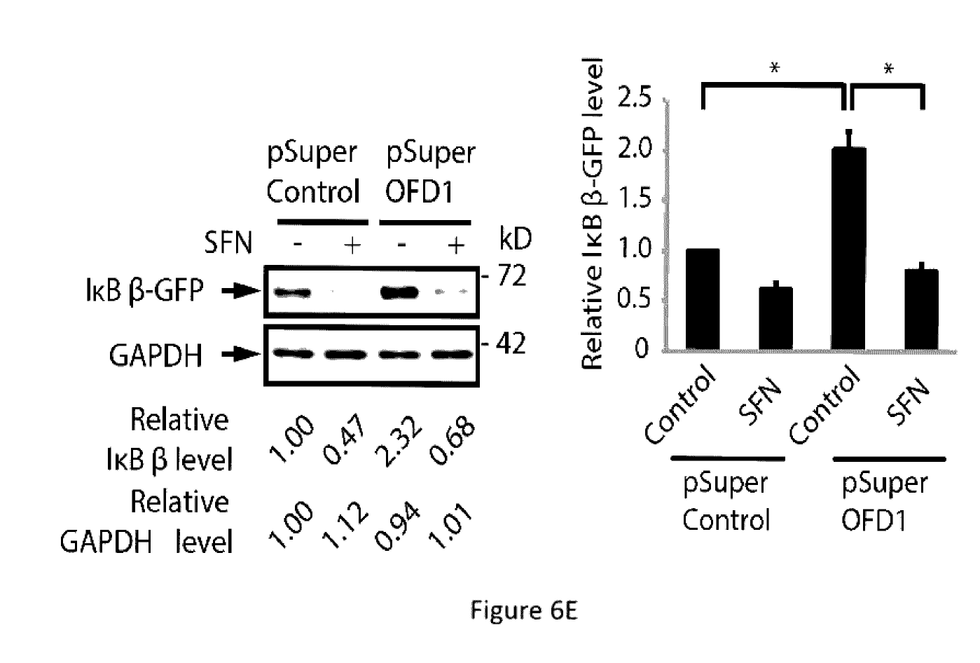

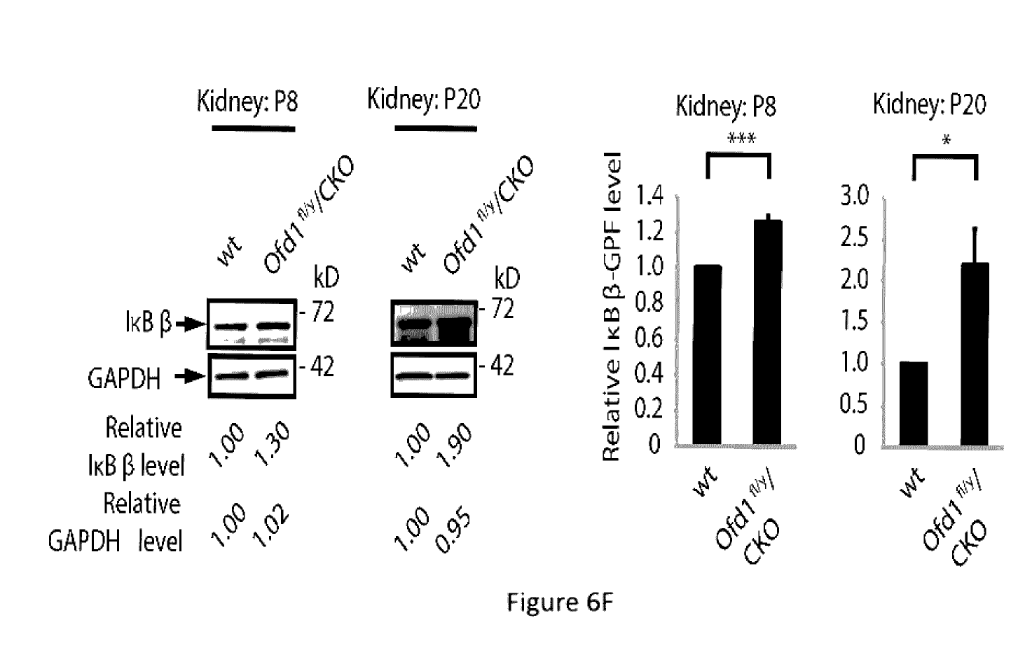

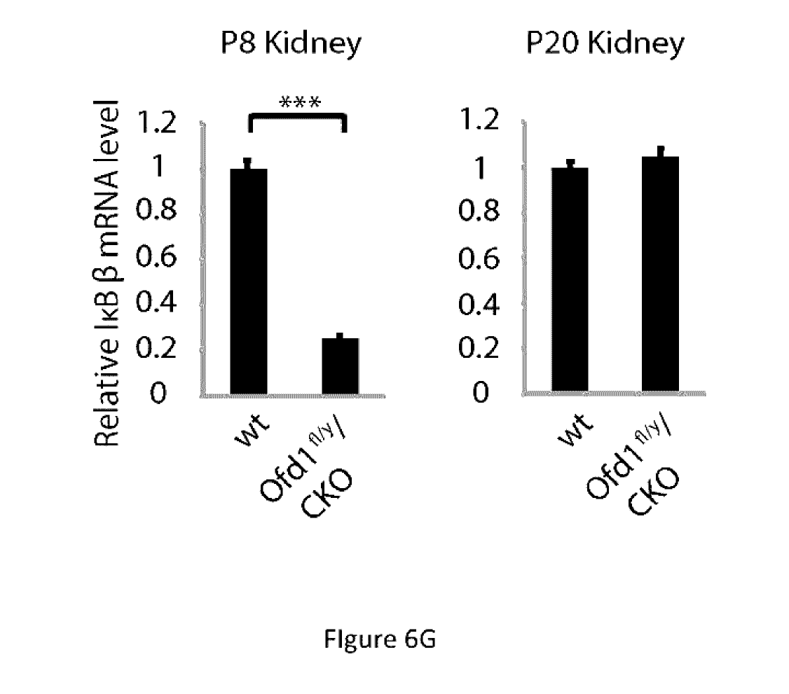

HEK-293-FT cells were transfected with an NF-κB luciferase reporter plasmid containing three copies of the KB response elements of the murine MHC class I promoter (3X-κB-L). Cells stimulated by TNF-α and cotransfected with the pSuperBBS4, pSuperBBS1, and pSuperOFD1 plasmids displayed a 55%, 53%, and 72% reduction in NF-κB activity, respectively, compared with that of control cells; incubation with SFN for 6 hours restored NF-κB activity (FIG. 6, A-C). In cells with depleted basal body proteins, the direction of the NF-κB activity change (suppressive signaling) is opposite that of Wnt and Notch activity change (excessive signaling) (5, 22). This inverse relationship is consistent with the model proposed herein, since the predicted basal body-regulated proteasomal degradation substrates are IκBs, which are negative regulators, while β-catenin and NICD are positive regulators. Also consistent with the luciferase assays, accumulation was observed for GFP-tagged IκBβ in BBS4-, BBS1-, or OFD-suppressant cells, which can also be ameliorated by SFN treatment (FIGS. 6, D and E).

Finally, to probe the potential physiological relevance of the observed NF-κB data in vivo, the expression and protein levels of the proteasomal substrate IκBβ in mice lacking Ofd1 was examined. Immunoblot analysis showed no differences in IκBβ protein levels on protein lysates from E10.5 Ofd1^{Δ4-5/y} mutant embryos and controls (data not shown). Since loss of Ofd1 in mice results in prenatal lethality, Ofd1-f loxed mice (Ofd1^{fl}) were generated and crossed with a CAG-Cre-ER™-inducible general deletor line to examine IκBβ protein levels in postnatal Ofd1 knockout mice. In Ofd1^{fl/y} CAG-Cre-ER™ (Ofd1^{fl/y} CKO) mice, Ofd1 inactivation was achieved at E18.5 by tamoxifen injection, and renal cysts were not observed at P8 (precystic stage). By P20, a replacement was observed of the renal parenchyma by cysts (cystic stage). Immunoblot analysis revealed an accumulation of IκBβ in protein lysates from kidney of Ofd1^{fl/y} CKO male mice compared with controls; crucially, this phenotype was evident in both precystic and cystic stages (FIG. 6F), suggesting that the accumulation of IκBβ is not a by-product of tissue disorganization and cystogenesis. At the precystic stage, mRNA levels of the IκBβ gene (Nfkbib) were lower in Ofd1^{fl/y} CKO mice compared with mRNA levels in WT animals (FIG. 6G), confirming that the accumulation of IκBβ is not due to increased mRNA transcription levels. Nfkbib mRNA levels were comparable between Ofd1^{fl/y} CKO and controls at the cystic stage (FIG. 6G).

The study of ciliary and basal body proteins has highlighted a complex role for this cellular region in the regulation of signaling pathways. These observations have raised critical questions, including whether dedicated signaling transduction machinery aggregate around the cilium and basal body. Several transduction components have been localized to the basal body and/or the ciliary axoneme, including Smoothened, GLI proteins, SUFU, β-catenin, adenomatosis polyposis coli (APC), and Notch3 (8, 10, 11, 13, 29). At the same time, a model in which the cilium facilitates each pathway independently is difficult to reconcile with the fact that (a) with the possible exception of the PCP transducer Fritz (17) and the Shh motor KIF7 (15, 16), none of the other 60-plus genes and proteins mutated in human ciliopathies are components of a specific paracrine signaling pathway; (b) the phenotype of ciliopathy patients is a mixture of defects more consistent with a context-specific pathway dysfunction; and (c) animal models ablated for specific ciliary or basal body proteins exhibit multiple signaling defects (1).

Without being limited to any one particular mechanism, the data presented herein suggest a simpler model, in which at least some basal body proteins play a role in signal transduction regulation by exerting their primary effect not on a given pathway per se, but by regulating context-dependent proteolytic degradation. The alternative would be that the observed phenotypes are the nonspecific consequence of generalized cellular malaise and that the observed rescue effects were reflective of broad improvement in the ability of the cell to eliminate proteins targeted for degradation. Taken together, the experiments provided herein favor the former model. Cells and embryos suppressed or ablated for each of BBS1, BBS4, and OFD1 had defects in proteasomal clearance of both reporter proteins and specific signaling components that included β-catenin, NICD, JAG1, GLI2, GLI3, SUFU, and IκBβ. It is also notable that the pronounced defects in proteasomal activity were concomitant with overt anatomical pathology in the murine models of disease described herein, such as in the retina of Bbs mice and the cystic kidneys of conditional Ofd1 animals, whereas no GFP accumulation was observed in Bbs mutant kidneys that, in the colony described herein, never exhibited cyst formation.

The robustness of the model was tested in four ways: (a) by predicting that NF-κB signaling, which requires proteasomal degradation (41), but has no known ciliary link, would be defective in the absence of the proteins of interest described herein; (b) by predicting the direction of the defect in NF-κB signaling; (c) by ameliorating the signaling defects for each tested pathway in vivothrough the chemical upregulation of proteasomal components; and (d) by demonstrating that in the absence of some basal body proteins, the centrosomal proteasome is partially depleted of the various regulatory subunits whose overexpression also has an ameliorating defect in vivo.

Notably, sucrose fraction sedimentation changes were observed in multiple proteasomal subunits in the absence of OFD1 or BBS4, arguing that ciliopathy phenotypes are unlikely to be driven by specific defects in only one subunit, consistent with the observations that the mice haploinsufficient for the subunit RPN10 are phenotypically normal, at least by gross pathology, while homozygous Rpn10^{-/-} mutants are embryonic lethal (42). While not desiring to be bound to any single mechanism of action, an attractive mechanism is one in which basal body proteins regulate the composition of multiple subunits in the proteasome holoenzyme in a context-dependent manner. This is known to occur during cellular stress (43), and it is plausible that ciliary signaling can have a similar effect.

The findings presented herein indicate that targeting the proteasome for compounds that increase its activity can have therapeutic applications for treatment of ciliopathic disorders.

### Methods

Immunoblotting.

Transfected cells and mouse tissues were lysed in modified RIPA buffer [150 mM sodium chloride, 50 mM Tris-HCl, pH7.4, 1% nonidet P-40, 0.1% sodium deoxycholate, 1 mM EDTA] with 1× proteasome inhibitor (Roche) and centrifuged at 4° C. for 15 minutes. Protein concentration was measured by Lowry assay using the DC Protein Assay Kit (Bio-Rad) on a DU 530 Life Science UV/Vis Spectrophotometer (Beckman Coulter). Total protein in each sample was separated by SDS-PAGE on 4% to 15% Mini-PROTEAN TGX Precast Gel (Bio-Rad) with a Spectra Multicolor Broad Range Protein Ladder (Fermentas) and transferred to an Immun-Blot PVDF Membrane (Bio-Rad). The membrane was blocked with 5% nonfat milk or 5% BSA (Sigma-Aldrich) and probed with the following commercial primary antibodies: anti-GAPDH (ab9484 from Abcam or sc-32233 from Santa Cruz Biotechnology Inc.); anti-GFP (sc-8334; Santa Cruz Biotechnology Inc. or ab13970; Abcam); anti-HSP90 (sc-7947; Santa Cruz Biotechnology Inc.); anti-GLI2 (AF3635; R&D Systems); anti-GLI3 (AF3690; R&D Systems); anti-SUFU (sc-10934; Santa Cruz Biotechnology Inc.); anti-Flag (F7425; Sigma-Aldrich); anti-catenin (sc-7199; Santa Cruz Biotechnology Inc.); anti-α-tubulin (T6199; Sigma-Aldrich); anti-NICD (ab8925; Abcam); anti-hsOFD1 (rabbit polyclonal antisera against human full-length OFD1 NM_003611); anti-mmOFD1 (rabbit polyclonal antisera against a portion of murine OFD1 NM_177429 aa 461-884); anti-BBS4 (AB15009; Millipore); anti-BBS1 (a166613; Abcam); anti-γ-tubulin (T7451; Sigma-Aldrich); anti-20S (NB600-1016; Novus); anti-PA28γ (NBP1-54587; Novus); anti-RPN10 (ab20239; Abcam); anti-RPN13 (H00011037-M01; Novus); anti-RPT2 (AP-107; Boston Biochem); anti-RPT6 (BML-PW9265; Enzo Life Sciences); anti-IκBβ (sc-9248; Santa Cruz Biotechnology Inc.); anti-Myc (m4439; Sigma-Aldrich); and anti-HA (ab16918; Abcam). Densitometric analysis was performed with Image Lab (Bio-Rad), Quantity One (Bio-Rad), or ImageJ 1.44p (NIH) software.

Immunohistochemistry.

Mouse eyes were fixed in 4% PFA, followed by immersion in sucrose (10%, 20%, and 30%) in PBS. With the lens removed, eyecups were embedded in Optimal Cutting Temperature Compound (Sakura) and flash frozen. Cryosections were blocked with 10% FBS in PBS and probed with primary antibodies anti-GFP (ab13970; Abcam) and anti-S-opsin (a gift from Jeremy Nathans, Johns Hopkins University, Baltimore, Md., USA), followed by secondary antibodies Alexa Fluor 488 IgG (Invitrogen) and Alexa Fluor 594 IgG (Invitrogen). Nuclei were stained with DAPI (Roche). Images were captured with a Nikon Eclipse 90i microscope.

Bioinformatic Analyses.

A subset of 271 transcripts were selected a that included: (a) transcripts mutated in human ciliopathies; (b) transcripts that, when mutated in animal models, give rise to ciliary dysfunction; (c) a group of transcripts found in at least to three of the available datasets of ciliary proteins (50); and (d) a subset of transcripts recently shown to be modulators of ciliogenesis and cilium length (51). The publicly available online tool (http://netview.tigem.it) was used to analyze the regulatory interactions among genes from genome-scale measurements of gene expression profiles (microarrays) (32).

ES Cell-Derived In Vitro Neural Differentiation.

The Ofd1-deficient E14Tg2A.4 KOES line was obtained from BayGenomics. Both WT and Ofd1 KOESs were maintained in an undifferentiated state by culture on a monolayer of mitomycin C-inactivated fibroblasts in the presence of leukemia-inhibiting factor (LIF). To induce neural differentiation, previously described protocols were followed (33). Briefly, 48 hours after ES cells were seeded on gelatin-coated plates, they were dissociated and plated on gelatin-coated plates at 1,000 cells/cm² on day 0 (TO). The culture medium for neuronal differentiation (serum-free KnockOut Serum Replacement-supplemented medium; Invitrogen) contained knockout DMEM supplemented with 15% KSR (Invitrogen), 2 mM L-glutamine, 100 U/ml penicillin-streptomycin, and 0.1 mM β-mercaptoethanol and was replaced daily during the differentiation process.

Cell Culture, DNA Transfection, and Drug Treatment.

HEK-293 or HEK-293-FT cells and human dermal fibroblasts were grown in DMEM (Invitrogen) containing 10% FBS (Invitrogen) and 2 mM L-glutamine (Invitrogen), hTERT-RPE1 cells in DMEM and Ham's F-12 Nutrient 1:1 mixture (DMEM/F-12; Invitrogen) with 10% FBS and 2 mM L-glutamine. FuGene6 Transfection Reagent (Roche) was used for transfection of expression constructs (including the Notch1-NICD expression construct, a gift from Nicholas Gaiano, Johns Hopkins University, Baltimore, Md., USA), shRNA-expressing plasmids, and luciferase reporter plasmids; then cells were cultured for 72 hours. Drug treatment was carried out at a final concentration of 10 μM SFN (Sigma-Aldrich) for 6 to 24 hours, 30 μm N-carbobenzoxyl- [symbol] -leucinyl- [symbol] -leucinyl- [symbol] -norleucinal (MG132; Calbiochem) for 5 hours, 20 μM lactacystin (EMD Bioscience) for 5 hours, and 50 ng/ml TNF-α (Sigma-Aldrich) for 12 hours.

Immunoprecipitation (IP).

For IP, approximately 1 mg of whole-cell, embryo, or tissue lysate was incubated with anti-OFD1, anti-Flag, or anti-GFP at 4° C. overnight, followed by incubation with protein G-coupled agarose beads (Santa Cruz Biotechnology Inc.) at 4° C. for 1 hour, or directly with anti-Flag M2 beads (A2220 Sigma-Aldrich). The beads were collected and washed with IP buffer (10% glycerol, 50 mM Tris-HCl [pH 7.5], 2.5 mM MgCl₂, 1% NP40, and 200 mM NaCl). Proteins conjugated with the beads were then denatured and separated from the beads by boiling at 95° C. to 100° C. for 5 minutes before processing for immunoblotting.

Affinity Purification of Proteasome Complex.

The 26S proteasome complex was purified following a previously described protocol (52) with modifications. Briefly, HEK-293-FT cells expressing stable HTBH-tagged hRPN11 (a gift from Lan Huang, University of California, Irvine, Calif., USA) were transfected with either pSuper control plasmid or pSuperBBS4 to knock down BBS4 expression. Seventy-two hours after transfection, cells were lysed in buffer A (100 mM NaCl, 50 mM Tris-HCl [pH 7.5], 10% glycerol, 2 mM ATP, 1 mM DTT, and 5 mM MgCl₂) with 1× proteasome inhibitor (Roche). Lysates were centrifuged at 4° C. for 15 minutes to remove cell debris. To purify proteasomes, an aliquot of the supernatant was incubated with streptavidin beads at 4° C. overnight to precipitate HTBH-RPN11. The beads were then washed with buffer A three times, followed by one washing with TEB buffer (50 mM Tris-HCl, pH 7.5 and 10% glycerol). Finally, the beads were incubated in TEB buffer containing 1% TEV protease at 30° C. for 1 hour, before SDS-PAGE and immunoblotting with anti-RPN10 (ab20239; Abcam).

Immunocytochemistry.

HEK-293 cells cultured on coverslips were fixed in methanol, blocked in normal goat serum (1:10 in PBS containing 5% BSA), and then probed with anti-RPN10 and anti-γ-tubulin, followed by secondary antibodies Alexa Fluor 488 IgG and Alexa Fluor 568 IgG. Finally, nuclei were visualized with Hoechst 33258 (Sigma-Aldrich). Images were captured with a Zeiss LSM 710 confocal microscope and analyzed with ImageJ 1.44p software.

Sucrose Gradient Sedimentation.

HEK-293-FT cells were transfected and treated with nocodazole (10 μg/ml) and cytochalasin B (5 μg/ml) for 1 hour at 72 hours after transfection. To collect cytoplasmic lysates, cells were harvested in lysis buffer (1 mM HEPES [pH 7.3], 0.5% NP-40, 0.5 mM MgCl₂, 0.1% β-ME, and 1× protease inhibitor), followed by centrifugation at 2,500 g for 10 minutes. After 10 mM HEPES and 5 U/ml DNase treatment for 30 minutes on ice, 1 ml of cytoplasmic lysates was layered on a discontinued sucrose gradient (70%, 50%, and 40% sucrose in the buffer containing 10 mM PIPES [pH 7.2], 0.1% NP-40, and 0.1% β-ME) and centrifuged for 1 hour at 195,000 g; 2% of lysates were kept before ultracentrifugation and served as an input. After ultracentrifugation, 13 fractions were collected and analyzed by immunoblotting.

Microinjection of Morpholinos, mRNA, and Sulforaphane.

Morpholinos against bbs1 (5′-CACACGTCCATCACTAACCAATAGC-3′; SEQ ID NO: 13), bbs4 (5′-CCGTTCTCATAGCGTCGTCCGCCAT-3′; SEQ ID NO: 14), and ofd1 (5′-ATCTTCTCTACTGCAACACACATAC-3′; SEQ ID NO: 15) were purchased from Gene Tools, LLC. Human RPN10, RPN13, and RPT6 mRNA were in vitro transcribed with a mMESSAGE mMACHINE SP6 Kit (Ambion). SFN was dissolved in DMSO (Sigma-Aldrich) at a stock concentration of 1 M and further diluted in water to 10 mM. The morpholino and mRNA or SFN were mixed, and a volume of 0.5 nl was microinjected.

Whole-Mount RNA In Situ Hybridization.

Zebrafish embryos were fixed overnight in 4% PFA at 4° C. Residual pigment was removed by bleaching with 3% H₂O₂/0.5% KOH. Whole-mount RNA in situ hybridization was performed with a digoxigenin-labeled anti-her4 RNA probe (a gift from Tohru Ishitani, Kyush University, Fukuoka, Kyushu, Japan) synthesized by in vitro transcription (Roche), followed by immunological detection with Anti-Digoxigenin-AP, Fab Fragments (Roche) and nitro blue tetrazolium/5-bromo-4-chloro-3-indolyl-phosphate staining (Roche).

Luciferase Reporter System Assays.

HEK-293-FT cells were seeded in 24-well plates at a density of 10⁴ cells/well. After 24 hours, cells were transfected with expression constructs, short-hairpin plasmids, and a 3X-κB-L reporter (a gift from Tom Gilmore, Boston University, Boston, Mass., USA) for NF-κB signaling. A pRL-SV40 plasmid expressing Renilla luciferase was used as an internal control. Seventy-two hours after transfection, cells were lysed with Passive Lysis Buffer (Promega). The luciferase activity of lysates was measured with the Dual Luciferase Reporter Assay System (Promega) on a FLUOstar Omega microplate reader (BMG LABTECH) and analyzed with MARS Data Analysis Software (BMG LABTECH).

Generation of Ofd1^{fl/y} CAG-Cre-ER™ Mice by Tamoxifen Injections.

Ofd1^{fl/+} females were crossed with CAG-Cre-ER™ mice, a general deletor Cre line in which Cre-ER is ubiquitously expressed after tamoxifen injection. Pregnant mothers were treated with a single i.p. injection of 100 μg tamoxifen/g of weight at E18.5. Tamoxifen (Sigma-Aldrich) was diluted in 10% ethanol and 90% sesame oil at a final concentration of 10 mg/ml. Quantification of Ofd1 inactivation was assessed by quantitative RT-PCR (qRT-PCR) (data not shown).

Real-Time qRT-PCR Analyses.

Total RNA was isolated with TRIzol (Invitrogen). The cDNA was synthesized from 5 μg of total RNA using SuperScript III (Invitrogen). qRT-PCR was performed with Power SYBR Green PCR Master Mix (Applied Biosystems) on a 7900HT Fast Real-Time PCR System (Applied Biosystems). The primers Nfkbib forward (5′-TTGGCTACGTCACTGAGGATG-3′; SEQ ID NO: 16) and Nfkbib reverse (5′-GCTCATGCTGATGAATCACAGC-3′; SEQ ID NO: 17) were used to test mRNA levels of Nfkbib, while the primers ofd1 forward (5′-TGGCAGACCACTTACAAAGATG-3′; SEQ ID NO: 18) and ofd1 reverse (AGACTGGATGAGGGGTTAATC-3′; SEQ ID NO: 19) were used to examine the conditional knockout efficiency, and the primers gapdh forward (5′-TCTTCTGGGTGGCAGTGAT-3′; SEQ ID NO: 20) and gapdh reverse (5′-TGCACCACCAACTGCTTAGC-3′; SEQ ID NO: 21) were used as internal controls. Real-time data were collected and analyzed with the Sequence Detection System software package, version 2.3 (Applied Biosystems).

Statistics.

A one-tailed Student's t test was performed to compare the means of two populations. In the bar graphs, data represent the mean±SEM of multiple repeats (n≧3). A χ² test was performed to compare two populations with several subgroups of different proportions. Statistical significance of differences between samples are indicated by *P<0.05, **P<0.01, and ***P<0.001. A P value less than 0.05 was considered significant.

### Example 2

### UPS Pathway and Individual Genes as Targets for Treatment of Ciliopathy Disorders

In a separate approach from the experiments described above, an in vivo genome-wide siRNA screening assay was performed to identify genes whose suppression can rescue the aberrant signaling transduction caused by loss of ciliary function. In this siRNA assay, USP35, a deubiquitinase that acts as a negative regulator of the ubiquitin-proteasome-system (UPS), was identified, as well as 12 other targets. This observed rescue effect seen in cells was then further tested in an animal model for 6 of the 13 identified targets. Specifically, a zebrafish model was used to recapitulate pathogenic features in patients. Depletion of 5 of the 6 targets in the zebrafish model, including USP35, was demonstrated to ameliorate the cerebellar and renal defects in the ciliary deficient animal model.

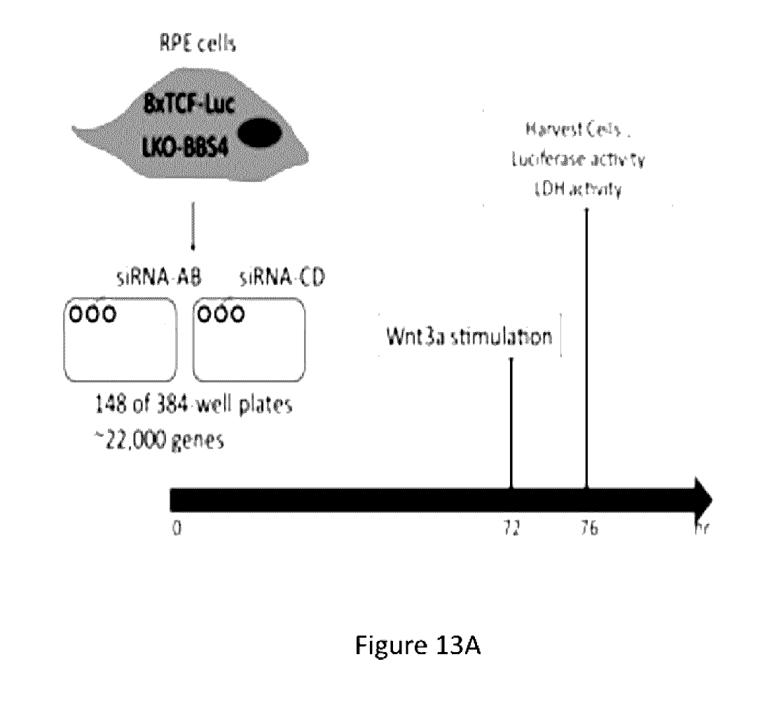

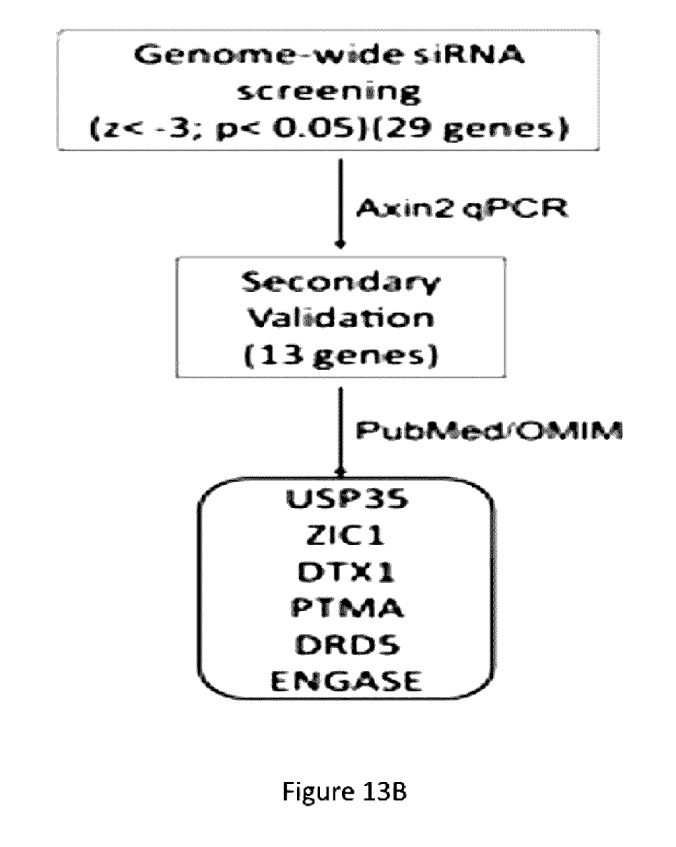

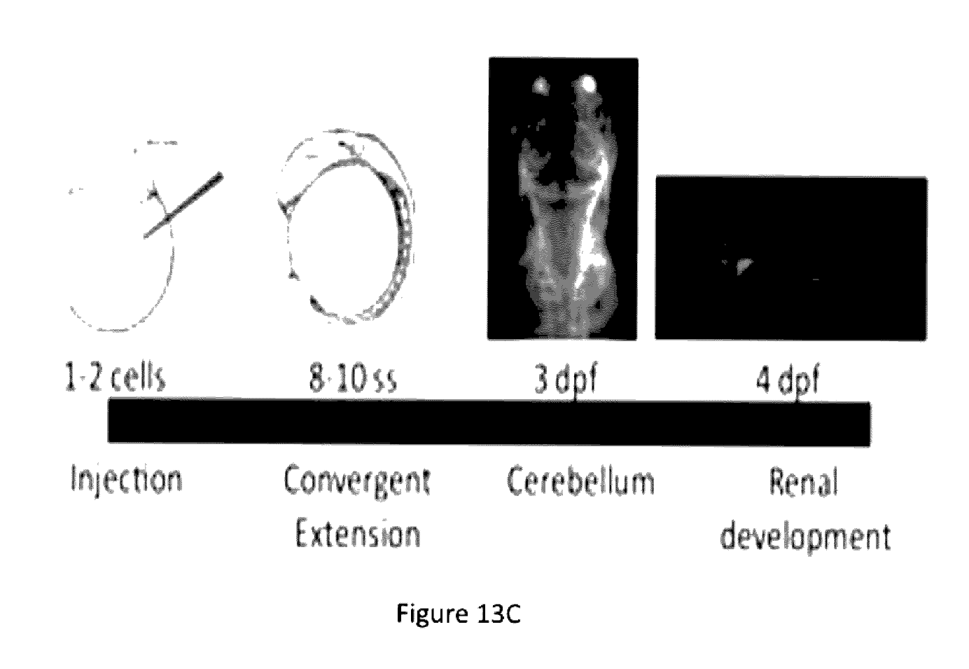

Specifically, a genome-wide RNAi screening was designed and executed with the to aim of isolating genes whose suppression can rescue hyper Wnt/3-cat signaling caused by loss of BBS4 (a well-known ciliary gene). An RPE (human retinal pigmentosa epicedium) cell line was generated that stably expresses 1) shRNA against BBS4 expression and 2) luciferase reporter possessing 8 TCF-binding sites (FIG. 13A).

Cells were transfected with Qiagen human whole-genome siRNA library, targeting approximately 22,000 genes in 384-well format. The library can be used as two half libraries, in which four non-related siRNAs (two siRNA in each half library) are designed to suppress the expression of one gene. 72 hours post transfection, cells were stimulated with Wnt3a for another four hours. Finally, cells were harvested and subjected for luciferase and LDH (for cell viability) assays. With the notion that some siRNAs may target to the genes that influence cell viability, leading to the identification of false suppressors, luciferase readout of each experiment was normalized with LDH activity to minimize errors caused by the variation of cell viability.

In this primary screening, 29 genes were identified that can significantly (z<−3; p<0.05) reduce the hyper activation of the Wnt/3-cat signaling. To validate these hits further, Axin2 qPCR was performed in the same cell type used for the primary screening with different source of siRNA (Dharmacon); 13 of 29 genes passed the secondary validation.

These genes are listed in Table 1.

TABLE 1
Genes identified as suppressors of Bbs4 loss of function phenotypes and
confirmed by the 2^{nd} screen.

| Gene | |
|---|---|
| Name | Gene Description |
| DTX1 | deltex homolog 1 (Drosophila) |
| PITPNM2 | phosphatidylinositol transfer protein, membrane-associated 2 |
| RHOXF1 | Rhox homeobox family, member 1 |
| DRD5 | dopamine receptor D5 |
| ENTPD6 | ectonucleoside triphosphate diphosphohydrolase 6 (putative |
| | function) |
| C14orf166 | chromosome 14 open reading frame 166 |
| PCF11 | PCF11, cleavage and polyadenylation factor subunit, homolog |
| | (S. cerevisiae) |
| TEX36 | testis expressed 36 |
| ZIC1 | Zic family member 1 (odd-paired homolog, Drosophila) |
| PTMA | prothymosin, alpha (gene sequence 28) |
| ENGASE | endo-beta-N-acetylglucosaminidase |
| TDRD12 | tudor domain containing 12 |
| USP35 | ubiquitin specific peptidase 35 |

After browsing PubMed and OMIM database, six of the genes from Table 1 were chosen for in vivo assay in a zebrafish model (FIGS. 13B and 13C).

Among the genes in Table 1, USP35 was particularly notable; it encodes an ubiquitin peptidase, which acts as a negative regulator in UPS (ubiquitin proteasome system)-mediated protein degradation. This gene target was of particular interest given the recent discovery described herein above of a connection between defective UPS and to ciliopathies, wherein ciliary depletion reduces proteasome degradation leading to the accumulation of signaling effector. Therefore, without desiring to be limited to any one particular mechanism of action, it was hypothesized that inhibition of USP35 can promote proteasome-dependent protein degradation, facilitating the clearance of signaling molecules.

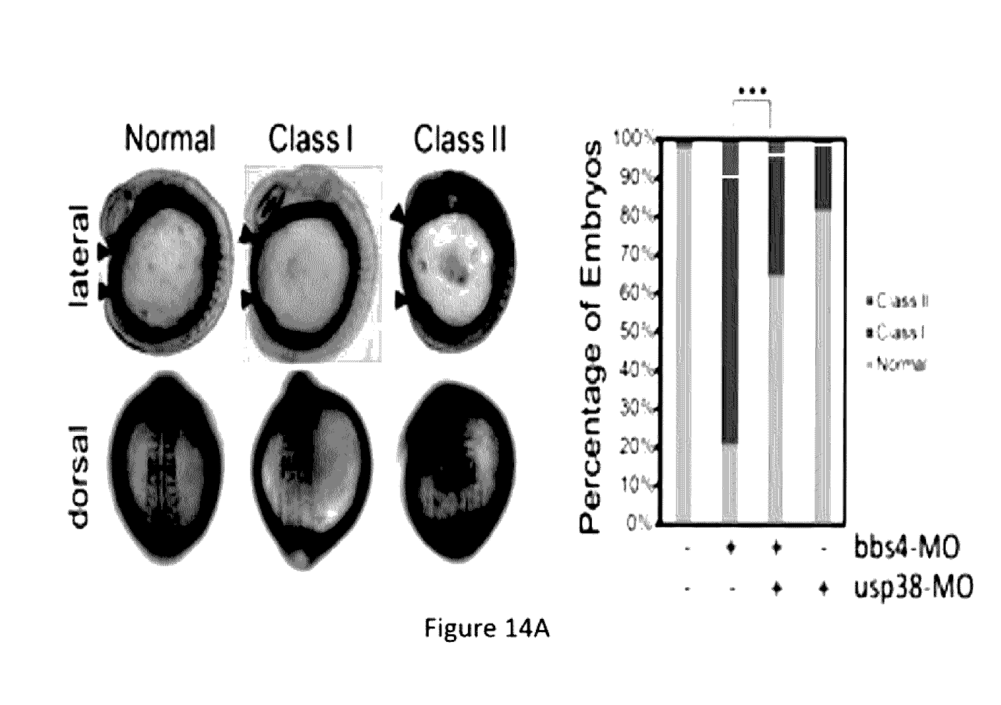

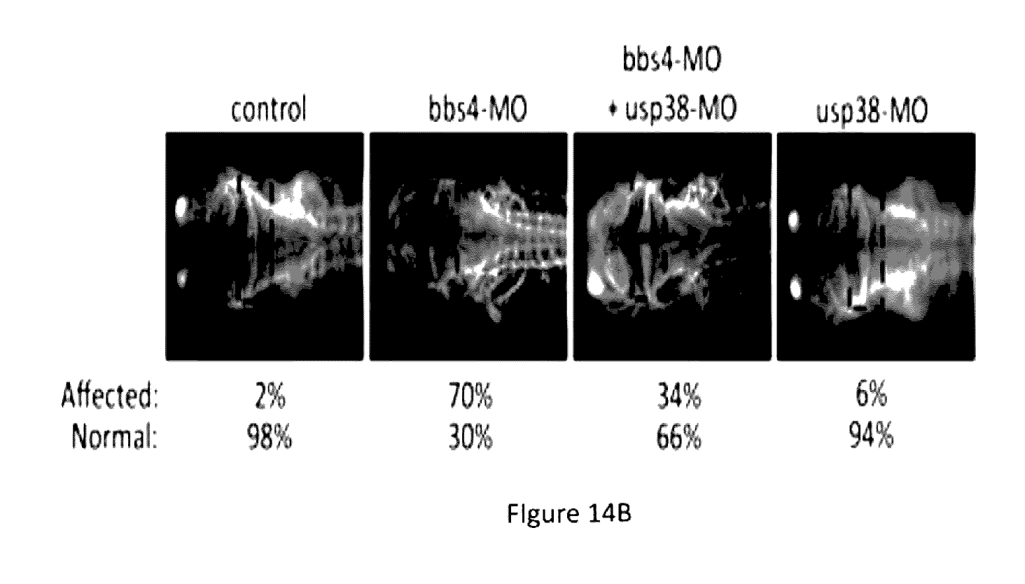

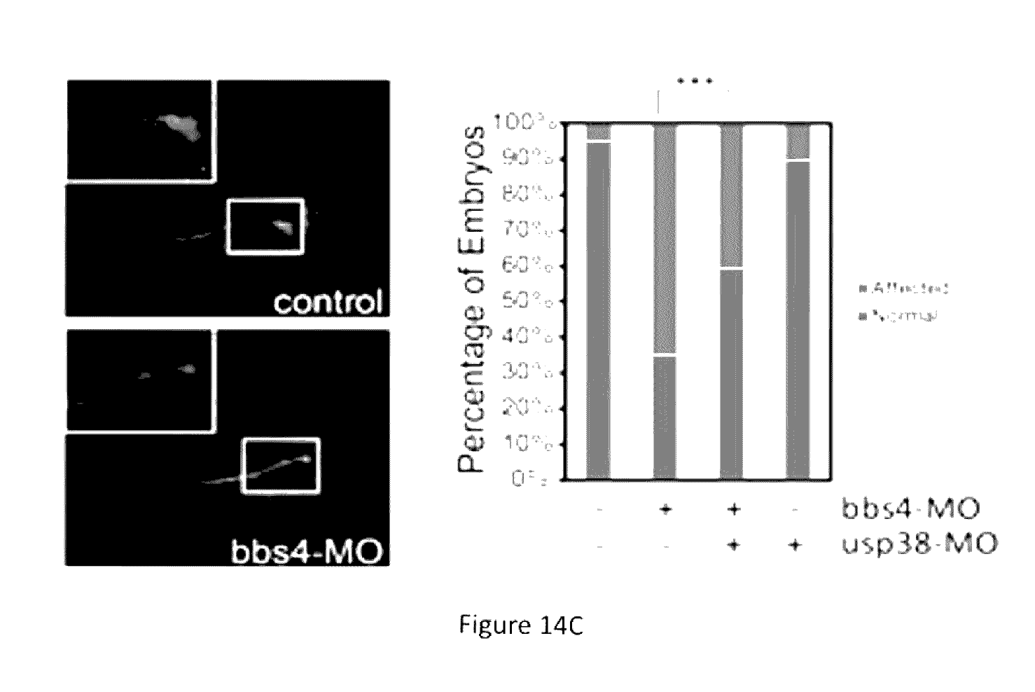

To test this hypothesis, an experiment was designed to test whether suppression of usp38 (the zebrafish ortholog of USP35) can rescue bbs4-depletion induced CE (convergent extension) defects, a phenotype caused by hyper activation of Wnt/3-cat pathway during gastrulation. The resulting data indicate that while depletion of bbs4 caused CE defects in ˜80% embryos, co-injection of a usp38-morpholino (usp38-MO) reduced significantly the incidence of pathology and severity in the embryos (30% in Class I and 5% in Class II; FIG. 14A). These data indicate that suppression of usp38 is able to ameliorate the hyper activation of Wnt/3-cat pathway, consistent with the in vivo screen.

Next phenotypic readout directly relevant to the clinical findings in ciliopathy patients was used to test the rescue efficiency of usp38 suppression. Identical dosages of MO as previous experiments were injected and 3 dpf (days post of fertilization) zebrafish larvae were collected to examine the morphologic development of the cerebellum. As shown in FIG. 14B, 70% of bbs4 morphants displayed organizational defects in the cerebellum abnormality, as visualized by acetylated-tubulin immunostaining of neuronal axons. Similar to the CE assay, significant rescue was observed as a result of usp38 suppression: the percentage of affected cerebellum was reduced to 34% by co-injection of usp38-MO (FIG. 14B).

In addition, as renal cyst formation is a major common feature in ciliopathy patients (and a key contributor to morbidity and mortality), the renal development was examined in 4 dpf zebrafish larva to evaluate the effect of usp38 suppression. In this model, bbs4 morphants consistently displayed atrophy and deficient convolution in proximal tubules, a phenotype reminiscent of renal immaturity reported in BBS patients and one likely source of cysts. Consistent with the CE and cerebellum studies described herein, the data showed that suppression of usp38 ameliorates the renal defects in the bbs4 morphant (FIG. 14C).

Taken together, the findings described herein demonstrate the efficiency of usp38 suppression to rescue the ciliopathy related phenotypes in zebrafish model. This indicates attenuation of USP35 activity in patients with ciliopathy disorders to improve disease prognosis.

Similar to USP35, the ZIC1 gene identified in the secondary screen was also tested in the zebrafish embryo model for CE rescue, cerebellum rescue, and renal rescue. In addition, each of the 4 gene targets DTX1, PTMA, DRD5, and ENGASE identified in the secondary screen was also tested in the zebrafish embryo model for CE rescue. The results are shown below in Table 2. The data demonstrate the ability of zic1, ptma, drd5, and engase suppression to rescue the CE phenotype in zebrafish model. This indicates attenuation of these gene product activities in patients with ciliopathy disorders to improve disease prognosis.

TABLE 2
Targets tested in zebrafish embryo model for CE rescue,
cerebellum rescue, and renal rescue.

| Knock-down (MO | | Cerebellum | Renal |
|---|---|---|---|
| or CRISPR/Cas9) | CE rescue | rescue | rescue |
| USP35 | Y | Y | Y |
| ZIC1 | Y | Y | Y |
| DTX1 | N | NT | NT |
| PTMA | Y | NT | NT |
| DRD5 | Y | NT | NT |
| ENGASE | Y | NT | NT |

Y—yes;
N—no;
NT—not tested

### REFERENCES

1. Hildebrandt F, Benzing T, Katsanis N. Ciliopathies. N Engl J Med. 2011; 364(16):1533-1543.
2. Eggenschwiler J T, Anderson K V. Cilia and developmental signaling. Annu Rev Cell Dev Biol. 2007; 23:345-373.
3. Lancaster M A, Gleeson J G. Cystic kidney disease: the role of Wnt signaling. Trends Mol Med. 2010; 16(8):349-360.
4. Ocbina P J, Tuson M, Anderson K V. Primary cilia are not required for normal canonical Wnt signaling in the mouse embryo. PLoS One. 2009; 4(8):e6839.
5. Gerdes J M, et al. Disruption of the basal body compromises proteasomal function and perturbs intracellular Wnt response. Nat Genet. 2007; 39(11):1350-1360.
6. Lancaster M A, Schroth J, Gleeson J G. Subcellular spatial regulation of canonical Wnt signalling at the primary cilium. Nat Cell Biol. 2011; 13(6):700-707.
7. Simons M, et al. Inversin, the gene product mutated in nephronophthisis type II, functions as a molecular switch between Wnt signaling pathways. Nat Genet. 2005; 37(5):537-543.
8. Corbit K C, et al. Kif3a constrains β-catenin-dependent Wnt signalling through dual ciliary and non-ciliary mechanisms. Nat Cell Biol. 2008; 10(1):70-76.
9. Oh E C, Katsanis N. Context-dependent regulation of Wnt signaling through the primary cilium. J Am Soc Nephrol. 2013; 24(1):10-18.
10. Corbit K C, Aanstad P, Singla V, Norman A R, Stainier D Y, Reiter J F. Vertebrate Smoothened functions at the primary cilium. Nature. 2005; 437(7061):1018-1021.
11. Haycraft C J, Banizs B, Aydin-Son Y, Zhang Q, Michaud E J, Yoder B K. Gli2 and Gli3 localize to cilia and require the intraflagellar transport protein polaris for processing and function. PLoS Genet. 2005; 1 (4):e53.
12. Schneider L, et al. PDGFRalphaalpha signaling is regulated through the primary cilium in fibroblasts. Curr Biol. 2005; 15(20):1861-1866.
13. Ezratty E J, Stokes N, Chai S, Shah A S, Williams S E, Fuchs E. A role for the primary cilium in Notch signaling and epidermal differentiation during skin development. Cell. 2011; 145(7):1129-1141.
14. Cano D A, Murcia N S, Pazour G J, Hebrok M. Orpk mouse model of polycystic kidney disease reveals essential role of primary cilia in pancreatic tissue organization. Development. 2004; 131(14):3457-3467.
15. Dafinger C, et al. Mutations in KIF7 link Joubert syndrome with Sonic Hedgehog signaling and microtubule dynamics. J Clin Invest. 2011; 121(7):2662-2667.
16. Putoux A, et al. KIF7 mutations cause fetal hydrolethalus and acrocallosal syndromes. Nat Genet. 2011; 43(6):601-606.
17. Kim S K, et al. Planar cell polarity acts through septins to control collective cell movement and ciliogenesis. Science. 2010; 329(5997):1337-1340.
18. Wigley W C, et al. Dynamic association of proteasomal machinery with the centrosome. J Cel Biol. 1999; 145(3):481-490.
19. Fabunmi R P, Wigley W C, Thomas P J, DeMartino G N. Activity and regulation of the centrosome-associated proteasome. J Biol Chem. 2000; 275(1):409-413.
20. Li J, et al. USP33 regulates centrosome biogenesis via deubiquitination of the centriolar protein CP110. Nature. 2013; 495(7440):255-259.
21. Mahuzier A, et al. Dishevelled stabilization by the ciliopathy protein RpgriplI is essential for planar cell polarity. J Cell Biol. 2012; 198(5):927-940.
22. Itoh K, Jenny A, Mlodzik M, Sokol S Y. Centrosomal localization of Diversin and its relevance to Wnt signaling. J Cell Sci. 2009; 122(pt 20):3791-3798.
23. Chitnis A B. The role of Notch in lateral inhibition and cell fate specification. Mol Cell Neurosci. 1995; 6(6):311-321.
24. Lai E C, Deblandre G A, Kintner C, Rubin G M. Drosophila neuralized is a ubiquitin ligase that promotes the internalization and degradation of delta. Dev Cell. 2001; 1 (6):783-794.
25. Kopan R. All good things must come to an end: how is Notch signaling turned off? Sci STKE. 1999; 1999(9):PE1.
26. Aza-Blanc P, Ramirez-Weber F A, Laget M P, Schwartz C, Komberg T B. Proteolysis that is inhibited by hedgehog targets Cubitus interruptus protein to the nucleus and converts it to a repressor. Cell. 1997; 89(7):1043-1053.
27. Pan Y, Bai C B, Joyner A L, Wang B. Sonic hedgehog signaling regulates Gli2 transcriptional activity by suppressing its processing and degradation. Mol Cell Biol. 2006; 26(9):3365-3377.
28. Chen M H, et al. Cilium-independent regulation of Gli protein function by Sufu in Hedgehog signaling is evolutionarily conserved. Genes Dev. 2009; 23(16):1910-1928.
29. Ross A J, et al. Disruption of Bardet-Biedl syndrome ciliary proteins perturbs planar cell polarity in vertebrates. Nat Genet. 2005; 37(10):1135-1140.
30. Ferrante M I, et al. Convergent extension movements and ciliary function are mediated by ofd1, a zebrafish orthologue of the human oral-facial-digital type 1 syndrome gene. Hum Mol Genet. 2009; 18(2):289-303.
31. Lindsten K, Menendez-Benito V, Masucci M G, Dantuma N P. A transgenic mouse model of the ubiquitin/proteasome system. Nat Biotechnol. 2003; 21 (8):897-902.
32. Belcastro V, et al. Transcriptional gene network inference from a massive dataset elucidates transcriptome organization gene function. Nucleic Acids Res. 2011; 39(20):8677-8688.
33. Fico A, Manganelli G, Simeone M, Guido S, Minchiotti G, Filosa S. High-throughput screening-compatible single-step protocol to differentiate embryonic stem cells in neurons. Stem Cells Dev. 2008; 17(3):573-584.
34. Singla V, Romaguera-Ros M, Garcia-Verdugo J M, Reiter J F. Ofd1, a human disease gene, regulates the length and distal structure of centrioles. Dev Cell. 2010; 18(3):410-424.
35. Pan Y, Wang B. A novel protein-processing domain in Gli2 and Gli3 differentially blocks complete protein degradation by the proteasome. J Biol Chem. 2007; 282(15):10846-10852.
36. Kwak M K, Cho J M, Huang B, Shin S, Kensler T W. Role of increased expression of the proteasome in the protective effects of sulforaphane against hydrogen peroxide-mediated cytotoxicity in murine neuroblastoma cells. Free Radic Biol Med. 2007; 43(5):809-817.
37. Lee B H, et al. Enhancement of proteasome activity by a small-molecule inhibitor of USP14. Nature. 2010; 467(7312):179-184.
38. Zaghloul N A, et al. Functional analyses of variants reveal a significant role for dominant negative and common alleles in oligogenic BardetBiedl syndrome. Proc Natl Acad Sci USA. 2010; 107(23):10602-10607.
39. Jiang Y J, Aeme B L, Smithers L, Haddon C, Ish-Horowicz D, Lewis J. Notch signalling and the synchronization of the somite segmentation clock. Nature. 2000; 408(6811):475-479.
40. Takke C, Dornseifer P, v Weizsacker E, Campos-Ortega J A. her4, a zebrafish homologue of the Drosophila neurogenic gene E(spl), is a target of NOTCH signalling. Development. 1999; 126(9):1811-1821.
41. Shih V F, Tsui R, Caldwell A, Hoffmann A. A single NFκB system for both canonical and non-canonical signaling. Cell Res. 2011; 21(1):86-102.
42. Hamazaki J, Sasaki K, Kawahara H, Hisanaga S, Tanaka K, Murata S. Rpn10-mediated degradation of ubiquitinated proteins is essential for mouse development. Mol Cell Biol. 2007; 27(19):6629-6638.
43. Hanna J, Meides A, Zhang D P, Finley D. A ubiquitin stress response induces altered proteasome composition. Cell. 2007; 129(4):747-759.
44. Jin H, et al. The conserved Bardet-Biedl syndrome proteins assemble a coat that traffics membrane proteins to cilia. Cell. 2010; 141(7):1208-1219.
45. Oeffner F, Moch C, Neundorf A, Hofmann J, Koch M, Grzeschik K H. Novel interaction partners of Bardet-Biedl syndrome proteins. Cell Motil Cytoskeleton. 2008; 65(2):143-155.
46. Garcia-Gonzalo F R, et al. Atransition zone complex regulates mammalian ciliogenesis and ciliary membrane composition. Nat Genet. 2011; 43(8):776-784.
47. Huang P, Schier A F. Dampened Hedgehog signaling but normal Wnt signaling in zebrafish without cilia. Development. 2009; 136(18):3089-3098.
48. Egner P A, et al. Bioavailability of Sulforaphane from two broccoli sprout beverages: results of a shortterm, cross-over clinical trial in Qidong, China. Cancer Prev Res (Phila). 2011; 4(3):384-395.
49. Shapiro T A, et al. Safety, tolerance, and metabolism of broccoli sprout glucosinolates and isothiocyanates: a clinical phase I study. Nutr Cancer. 2006; 55(1):53-62.
50. Inglis P N, Boroevich K A, Leroux M R. Piecing together a ciliome. Trends Genet. 2006; 22(9):491-500.
51. Kim J, et al. Functional genomic screen for modulators of ciliogenesis and cilium length. Nature. 2010; 464(7291):1048-1051.
52. Wang X, Chen C F, Baker P R, Chen P L, Kaiser P, Huang L. Mass spectrometric characterization of the affinity-purified human 26S proteasome complex. Biochemistry. 2007; 46(11):3553-3565.
53. Liu Y P, et al. Ciliopathy proteins regulate paracrine signaling by modulating proteasomal degradation of mediators. J Clin Invest. 2014; 124(5):2059-2070. doi:10.1172/JCI71898.

Any patents or publications mentioned in this specification are indicative of the levels of those skilled in the art to which the invention pertains. These patents and publications are herein incorporated by reference to the same extent as if each individual publication was specifically and individually indicated to be incorporated by reference.

One skilled in the art will readily appreciate that the presently described subject matter is well adapted to carry out the objects and obtain the ends and advantages mentioned, as well as those inherent therein. The present examples along with the methods described herein are presently representative of preferred embodiments, are exemplary, and are not intended as limitations on the scope of the invention. Changes therein and other uses will occur to those skilled in the art which are encompassed within the spirit of the invention as defined by the scope of the claims.

We claim:

1. A method of screening for a therapeutic molecule to treat a ciliopathy disorder, the method comprising:
measuring the activity of a ubiquitin-proteasome system (UPS)-mediated protein degradation in the presence and the absence of a candidate molecule, wherein an increase of the UPS-mediated protein degradation activity in the presence of the candidate molecule identifies the candidate molecule as a potential therapeutic molecule to treat a ciliopathy disorder.

2. The method of claim 1, wherein the UPS-mediated protein degradation activity is measured directly at the ubiquitin-proteasome holoenzyme.

3. The method of claim 2, wherein the candidate molecule comprises a small molecule or an antibody.

4. The method of claim 1, wherein the UPS-mediated protein degradation activity is measured indirectly via a cell-based or an animal-based model, wherein the cell-based or animal-based model comprises a silenced, reduced, or depleted expression of one or more basal body/ciliary genes comprising a BBS4 (Bardet-Biedl syndrome 4) gene, a BBS1 (Bardet-Biedl syndrome 1) gene, or an OFD1 (Oral-facial-digital syndrome 1) gene that results in reduced ubiquitin-proteasome system (UPS)-mediated protein degradation.

5. The method of claim 4, wherein the cell-based model comprises a human retinal pigmentosa epicedium cell line.

6. The method of claim 5, wherein the basal body/ciliary gene is the BBS4 gene.

7. The method of claim 6, wherein the human retinal pigmentosa epicedium cell line stably expresses a short hairpin RNA (shRNA) against BBS4 expression.

8. The method of claim 4, wherein the animal-based model comprises a morphant zebrafish embryo model having a bbs4 depletion-induced convergent extension (CE) defect, cerebellum organizational abnormality, and a renal development abnormality.

9. The method of claim 4, wherein the animal-based model comprises an Ofd1 knockout mouse model.

10. The method of claim 4, wherein the candidate molecule comprises a small molecule, an antibody, a RNA interference molecule (RNAi), a short hairpin RNA (shRNA), or a small interfering RNA (siRNA).

11. A method of screening for a therapeutic molecule to treat a ciliopathy disorder, the method comprising:
measuring the activity of a negative regulator of a ubiquitin-proteasome system (UPS) in the presence and the absence of a candidate molecule, wherein a decrease in the activity of the negative regulator of the UPS in the presence of the candidate molecule identifies the candidate molecule as a potential therapeutic molecule to treat a ciliopathy disorder.

12. The method of claim 11, wherein the negative regulator of the ubiquitin-proteasome system (UPS) comprises: a ubiquitin peptidase (USP35) gene product or an ortholog thereof, a Zic family member 1 (ZIC1) gene product or an ortholog thereof, a dopamine receptor D5 (DRD5) gene product or an ortholog thereof, a prothymosin alpha gene sequence 28 (PTMA) or an ortholog thereof, an endo-beta-N-acetylglucosaminidase (ENGASE) gene product or an ortholog thereof, a phosphatidylinositol transfer protein (PITPNM2) gene product or an ortholog thereof, a Rhox homeobox family member 1 (RHOXF1) gene product or an ortholog thereof, an ectonucleoside triphosphate diphosphohydrolase 6 (ENTPD6) gene product or an ortholog thereof, a chromosome 14 open reading frame 166 (C14orf166) gene product or an ortholog thereof, a cleavage and polyadenylation factor subunit homolog (PCF11) gene product or an ortholog thereof, a testis expressed 36 (TEX36) gene product or an ortholog thereof, or a tudor domain containing 12 (TDRD12) gene product or an ortholog thereof.

13. The method of claim 12, wherein the candidate molecule comprises a small molecule, an antibody, a RNA interference molecule (RNAi), a short hairpin RNA (shRNA), or a small interfering RNA (siRNA).

14. A method of screening for a therapeutic molecule to treat a ciliopathy disorder, the method comprising:
measuring the activity of a ubiquitin peptidase USP35 gene product, or an ortholog thereof, in the presence and the absence of a candidate molecule, wherein a decrease of the activity in the presence of the candidate molecule identifies the candidate molecule as a potential therapeutic molecule to treat a ciliopathy disorder.

15. A method of screening for a therapeutic molecule to treat a ciliopathy disorder, the method comprising:
measuring the activity of a Zic family member 1 (ZIC1) gene product or an ortholog thereof, in the presence and the absence of a candidate molecule, wherein a decrease of the activity in the presence of the candidate molecule identifies the candidate molecule as a potential therapeutic molecule to treat a ciliopathy disorder.
